(19) United States
(12) Patent Application Publication
(10) Pub. No.: US 2025/0205358 A1
(43) Pub. Date: Jun. 26, 2025

(54) PHOTOACTIVATABLE NANOPARTICLES AND USES THEREOF AS AGENTS FOR IMMUNOTHERAPY COMBINATIONS

(71) Applicant: Board of Regents, The University of Texas System, Austin, TX (US)

(72) Inventors: Girgis Obaid, Allen, TX (US); Chanda Bhandari, Richardson, TX (US); Nimit Shah, Richardson, TX (US)

(73) Assignee: Board of Regents, The University of Texas System, Austin, TX (US)

(21) Appl. No.: 18/984,124

(22) Filed: Dec. 17, 2024

Related U.S. Application Data
(60) Provisional application No. 63/612,499, filed on Dec. 20, 2023.

(51) Int. Cl. A61K 47/69; A61K 41/00; A61K 45/06; A61K 47/68; A61P 35/00; C07K 16/28

(52) U.S. Cl. CPC A61K 47/6913; A61K 41/0071; A61K 45/06; A61K 47/6849; A61P 35/00; C07K 16/2827

(57) ABSTRACT

Photoactivatable nanoparticles for simultaneous PD-L1 immune checkpoint targeting and blocking are capable of modulating tumor stroma, promoting self-delivery, improving tumor growth inhibition, and improving survival outcomes even with just a single priming dose. Light activation of the nanoparticles disrupts tumor collagen, reduces tumor fibroblasts, and promotes their self-delivery in vitro and in vivo which correlates with overall survival in mice. This demonstrates their ability to overcome the most significant barrier in nanoparticle delivery in PDAC tumors.

This application claims priority to U.S. Provisional Patent Application Ser. No. 63/612,499, entitled “Photoactivatable Nanoparticles and Uses Thereof as Agents for Immunotherapy Combinations,” filed Dec. 20, 2023, the entire contents of which are hereby incorporated by reference.

This invention was made with government support under Grant No. R00 CA215301 awarded by the National Institutes of Health. The government has certain rights in the invention.

This disclosure pertains to photoactivatable nanoparticles useful in immunotherapy based combinations, including but not limited to chemo-immunotherapy and immuno-radiotherapy.

Pancreatic Ductal Adenocarcinoma (PDAC) is a deadly cancer with a 5-year survival rate of 11%. Liposomes (namely Onivyde, formulation of irinotecan) are approved for metastatic PDAC. Although tireless efforts are dedicated to improving patient survival rates, PDAC remains to be the third leading cause of death in the US. Of all PDAC diagnoses, 80% of cases are unresectable, which exhibit local or distant metastases. The current standard of care for PDAC patients includes two combination regimens; gemcitabine plus nab-paclitaxel (Abraxane; nanoparticle drug) and FOLFIRINOX. However, overall survival is only 8.5 months and 11.1 months, respectively. Onivyde extends median survival up to only 6.1 months. The poor prognoses in PDAC are in part due to desmoplasia, a fibrotic stromal reaction that leads to elevated deposition of extracellular matrix (ECM). Desmoplasia leads to limited drug delivery through the tumor parenchyma, which is especially problematic for larger therapies, such as nanoparticle and nanoliposomal drug formulations. Desmoplasia also contributes to chemoresistance, radioresistance, an immunosuppressive microenvironment, and pro-tumorigenic survival signaling. As such, there is still a significant need for major therapeutic advances that overcome the intratumor barrier to further improve survival in PDAC patients.

Currently, immunotherapy has been used for the management of several cancers, including solid tumors. Chemo-immunotherapy regiments are being evaluated in over 150 clinical trials for PDAC. Immune checkpoint inhibitors commonly used in these clinical trials include α-PD-1 and α-PD-L1 antibodies which target cytotoxic T cells and cancer cells, respectively, to enable cytotoxic T cell activity against tumors. PD-L1 is expressed on both tumor cells and host myeloid cells, such as macrophages and dendritic cells. PD-L1 expression in host myeloid cells is also found to be essential for effective responses to PD-L1 blockade. Importantly, PD-L1 is expressed in up to 90% of PDAC patients and primary PDAC cell lines. α-PD-L1 antibodies, such as durvalumab, atezolizumab, and avelumab, are therefore being used in over 30 clinical studies in combination with chemotherapy. However, even with the most successful chemo-immunotherapy clinical trial for PDAC (PRINCE), median survival was only 16.7 months. The poor efficacy of immunotherapy in PDAC is primarily attributed to immunosuppression and immunologic heterogeneity. Immunologically cold PDAC tumors typically contain weakly immunogenic cancer cells and immunosuppressive cytokines, such as Tumor Necrosis Factor-α (TNF-α), Interleukin-6 (IL-6), and Interleukin-1 β (IL-1β). Furthermore, the dense and highly aligned collagen fibers present in PDAC tumors are speculated to reduce the contact, and thus killing, of cancer cells by cytotoxic T lymphocytes. Additionally, conventional wisdom suggests that immune responsiveness in PDAC is dependent on microsatellite instability; however, microsatellite instability is found in only 1-2% of all PDAC cases. Therefore, there remains a critical need of more transformative approaches that can provide meaningful immunotherapy responses in PDAC.

Photodynamic therapy (PDT) is a light-activated, anti-cancer modality that involves the use of photochemical reactions mediated by photosensitizers (PSs). Antibody-targeted photodynamic therapy, also known as photoimmunotherapy, is in Phase III trials for head and neck cancer in the US (ClinicalTrials.gov, Identifiers: NCT03769506) and has been approved in Japan. α-PD-L1 antibodies have also been shown to be effective vehicles for photoimmunotherapy agents in ovarian and lung tumors. However, the low PS-to-antibody ratio can significantly limit the efficacy of photoimmunotherapy. This limitation can be effectively overcome by encapsulating the PSs into high payload nanoliposomal formulations, which are then surface functionalized with antibodies to target them to tumor receptors. The nanoliposomal formulation of the PS benzoporphyrin derivate (BPD), also known as Visudyne, has already been shown to induce >4 cm of necrosis in patient PDAC tumors following 690 nm light activation when multiple optical fibers are used to deliver light. Pre-clinically, the tumor microenvironment can be primed using sub-curative doses of PDT (Photodynamic Priming; PDP) to improve the tumor accumulation and intratumoral distribution of chemotherapy, full-length antibodies and various nanoparticles. However, all such approaches have been attempted using two separate agents: a PDP agent containing the PS and the therapeutic entity. Using multiple agent administration will inevitably complicate clinical translation of PDP for improving drug delivery to solid tumors. No self-delivery using PDP of any single-agent (nano)therapeutic containing a PS has been attempted. It has been shown that nanoparticle delivery in solid tumors correlates with tumors responses in patients.

PDP remediates desmoplasia in in vivo models of PDAC. PDT of adventitial fibroblasts was shown to decrease their activation, migration, and proliferation. Furthermore, PDP of human vocal fold fibroblasts was found to deplete ECM proteins, such as collagen type I α2 chains, collagen type III α1 chains, fibronectin, and elastin while also increasing matrix remodeling enzymes (matrix metallopeptidase 1). Photodynamic stromal depletion of 3D PDAC models improves the delivery of therapeutic nanoparticles that were administered as separate agents. Improved nanoparticle delivery coincides with softening of a Matrigel matrix and selective depletion of stromal cells. Moreover, fluence is important for the photomodulation of collagen hydrogels using riboflavin. Below 25 J/cm², the collagen matrix was stiffened and above 25 J/cm² it was softened. PDP using EGFR-targeted nanoliposomal formulations of lipid-conjugated BPD (BPD-PC) can remediate desmoplasia in subcutaneous and orthotopic PDAC models by reducing collagen density and alignment. Interestingly, disruption of orthotopic PDAC tumor collagen alignment by light activation of these constructs correlates significantly with survival benefit. While EGFR targeted BPD-PC liposomes encapsulating irinotecan doubled overall survival following light activation, without targeting no tumor growth delay was observed.

PDP using BPD-PC liposomes converts immunologically ‘cold’ PDAC tumor spheroids to immunologically “hot” ones. PDP-mediated release of damage-associated molecular patterns (DAMPs), such as calreticulin (CRT), heat shock protein 60 (HSP 60), heat shock protein 70 (HSP 70), and high mobility group box 1 (HMGB1) can induce both innate immunity and T cell-mediated adaptive immunity against light-treated tumors. Naturally, this paves the way for combining PDP with immune checkpoint inhibitors. PDP has been combined with α-PD-L1 antibodies for PDP-triggered immune activation and α-PD-L1 mediated immune checkpoint inhibition however most of these strategies require multiple cycles of treatment. Recently, it was shown that a single protocol PDP improves the delivery of α-PD-L1 antibodies and induces immunogenicity in head and neck cancer. Although promising, antibody-based immune checkpoint inhibitors are associated with an increased risk of adverse immune-related adverse events in patients. As such, there is a need to also potentiate immunotherapy responses in order to lower the required doses of immune checkpoint inhibitors and mitigate toxicity.

α-PD-L1 antibodies have been co-encapsulated with chlorin e6 (Ce6) PS in a nanoformulation via an assembly strategy to develop a nanoconstruct for PDT-enhanced immunotherapy. The PDT-based regimen was found to control primary tumors and metastases and prevent relapse following light activation. However, the α-PD-L1 antibodies were designed to separate from the nanoconstruct before they reached the tumor, and therefore, no molecular targeted delivery through PD-L1, such as that described herein, was attempted. The photochemical internalization (light induced intracellular delivery; PCI) of an α-PD-L1-immunotoxin was demonstrated, which enhanced the cytotoxicity dramatically in an in vitro model of breast cancer. However, the PS for PCI and the α-PD-L1-immunotoxin were administered separately. PD-L1 aptamer functionalized metal-organic framework nanoparticles were developed for combined PDT and immunotherapy of colon cancer. This combination caused regression of both illuminated and non-illuminated tumors by inducing tumor-specific and systemic anti-tumor immunity. Although highly promising, aptamers are not approved for immunotherapy, and therefore targeting and blocking immune checkpoints using α-PD-L1 antibodies, such as that described herein, is more clinically translatable.

Bone marrow mesenchymal stem cell-derived nanovesicles were engineered to express α-PD-L1 antibodies to combine immunotherapy with photothermal therapy using indocyanine green (ICG). This strategy significantly enhanced the efficacy of photothermal ablation as well as the subsequent immune activation in murine melanoma model. Although promising, the passively loaded ICG has a propensity to leak out prematurely and compromise tumor tissue specificity. What is still needed is a stable, rapidly translatable photochemical lipid nanosystem, such as that described herein, for PDP and immunotherapy that also exhibits true molecular specificity towards tumor PD-L1.

### SUMMARY

The present disclosure relates generally to immune checkpoint targeted photoactivatable nanoparticles that exhibit self-delivery through solid tumors upon activation with light. The nanoparticles used herein can be any suitable nanoparticle carrier to which a photoactivatable feature and a feature for immune checkpoint targeting can be added. Any immune checkpoint can be targeted.

In certain embodiments, PD-L1 immune checkpoint targeted photoactivable liposomes (iTPALs) leverage PDP to promote self-delivery through solid PDAC tumors. The exemplary α-PD-L1 antibody targeted liposomes allow for tumor cell specific delivery and PDP and therapy. It is notable that a photoactivable nanoconstruct can exhibit self-penetrating properties through solid tumors upon activation with light. This is especially significant considering that desmoplasia presents a serious impediment in nanoparticle drug delivery through PDAC tumors, and many other solid tumors. The CT1BA5 tumor model used in examples in this disclosure is a syngeneic tumor line derived from a KPfC (Kras^{LSL-G12D}; Trp53^{fl/fl}; PDX^{Cre/+}) genetically engineered mouse model (GEMM) of PDAC. CT1BA5 tumors are highly aggressive and have a doubling time of 5.5 days, as calculated using established tumor growth models. This is significantly shorter than the doubling time of MIA PaCa-2+PCAF tumors (17.8 days) previously reported.

Photoactivation not only modulates the tumor environment but sensitizes the tumor tissue to respond better to immunotherapy, chemo, radio, and many other treatments. Preferred embodiments of the nanoparticle platform disclosed herein are intended to be used in conjunction with and incorporate multiple types of additional treatments, such as chemotherapy, small molecular weight inhibitors, targeted, drugs, nucleuic acids, mRNA, DNA, radiosensitizers, and the like.

Nanoparticle self-delivery by light activation can be by perforating tumor blood vessels and enhancing extravasation. Nanoparticle self-delivery by light activation can be by modulating other extracellular matrix molecules, such as fibronectin, hyaluronic acid, laminin, elastin etc. There is some evidence in the literature that suggests reactive oxygen species can also oxidize other types of extracellular matrix molecules

The construct described herein preferably integrates 4 salient features: (1) tumor cell-specific phototoxicity through immune checkpoint (for example, PD-L1) targeting, 2) immune checkpoint inhibition by blocking the relevant (for example, PD-1/PD-L1) axis, 3) light-triggered induction of immunogenic cell death, and 4) priming of tumor collagen and tumor fibroblasts to promote self-delivery through solid PDAC tumors 5) inhibit tumor growth and improve survival even with a single sub-curative priming dose. In preferred embodiments, this strategy benefits from high-payload delivery of the PS BPD-PC and from enhanced immune checkpoint blockade by multivalent effects of the surface-conjugated targeting feature (e.g. α-PD-L1 antibody). It also enables the future use of rational multi-agent co-encapsulation and co-delivery in PDAC models, with an emphasis on further improving the survival benefit that both remediating desmoplasia and eradicating distant metastases will provide.

### BRIEF DESCRIPTION OF THE DRAWINGS

FIG. 1 shows a schematic representation of the utility of PD-L1 immune checkpoint targeted photoactivable liposomes (iTPALs (17α)) according to preferred embodiments described herein.

FIG. 2A shows a schematic representation of preferred embodiments of targeted photoactivable liposomes (iTPALs) entrapping the BPD-PC PS on the hydrophobic bilayer and α-PD-L1 antibodies on the surface.

FIG. 2B shows conjugation of varying α-PD-L1 antibody-to-liposome ratios on iTPALs (17α).

FIG. 2C shows median BPD emission from iTPALs obtained from the flow cytometry analysis on the cellular uptake of different α-PD-L1 antibody-to-liposome ratios (0, 3, 9, 17, 27, 35 antibodies per liposome) at 1 h, 6 h, 9 h, 24 h and 48 h of incubation in CT1BA5 cells.

FIG. 2D shows a bar graph depicting the cellular uptake of iTPALs (17α) which shows a significant improvement on the cellular uptake of iTPALs (17α) at 9 h.

FIG. 2E shows percent improvement in cellular uptake of iTPALs with respect to untargeted PALs at different time points mentioned.

FIG. 2F shows percent improvement in iTPALs (17α) at 9 h in comparison to other iTPALs.

FIG. 3A shows hydrodynamic diameter of iTPALs (17α) over 8 days in storage.

FIG. 3B shows polydispersity indices of iTPALs (17α) over 8 days in storage.

FIG. 4A shows the generation of singlet oxygen by iTPALs (17α), PALs, and conventional liposomal BPD (Lipo-BPD), as confirmed by the decreased absorbance of the colorimetric singlet oxygen probe ADPA with increasing fluence of 690 nm light.

FIG. 4B shows the generation of singlet oxygen by iTPALs (17α), PALs, and conventional liposomal BPD (Lipo-BPD), as confirmed by the increased fluorescence of the fluorogenic singlet oxygen probe SOSG.

FIG. 4C shows hydroxyl radical and peroxynitrite anion generation by iTPALs (17α), PALs, and conventional liposomal BPD (Lipo-BPD, as measured using the fluorogenic probe HPF.

FIG. 4D shows area under curve of ADPA absorbance for iTPALs (17α), PALs, and conventional liposomal BPD (Lipo-BPD).

FIG. 4E shows area under curve of SOSG fluorescence emission for iTPALs (17α), PALs, and conventional liposomal BPD (Lipo-BPD).

FIG. 4F shows area under curve of HPF emission for iTPALs (17α), PALs, and conventional liposomal BPD (Lipo-BPD).

FIG. 5A shows fluorescence emission of Lipo-BPD, PALs and iTPALs (17α) in DPBS with increasing fluence of 690 nm irradiation.

FIG. 5B shows photobleaching rate kinetics of Lipo-BPD, PALs and iTPALs (17α).

FIG. 6A shows increase in PD-L1 expression with mIFN-γ on murine cell lines: LLC (Lewis lung carcinoma), CT1BA5 (PDAC), MC38 (colorectal cancer), AT84 (head and neck cancer), BMFA3 (PDAC) and ID8 (ovarian cancer).

FIG. 6B shows binding specificity of iTPALs (17α) to murine cancer cell lines, with respect to untargeted PALs.

FIG. 6C shows PD-L1 expression relative to the binding specificity of iTPALs (17α).

FIG. 7 shows confocal microscopy images of CT1BA5 cells using a 100× objective after 24 h incubation with iTPALs (17α) (left), with lysosomes labeled with LysoTracker™ Green DND-26 (middle), and the first two images merged to show regions of colocalization between iTPALs (17α) and lysosomes (right).

FIG. 8A shows representative confocal microscopy images showing cytotoxic T cell-mimicking microspheres tagged with recombinant mouse PD-1 binding to CT1BA5 cells (left), and pre-incubated with PALs (middle) and iTPALs (17α) (right).

FIG. 8B shows PD-1 microsphere binding to CT1BA5 cancer cells for PALs, iTPALs (17α), and free α-PD-L1.

FIG. 8C shows PD-1 microsphere binding to CT1BA5 cancer cells for iTPALs (17α) and iTPALs (35α).

FIG. 8D shows PD-1 microsphere binding to AT84 cancer cells for iTPALs (17α) and iTPALs (35α).

FIG. 9A shows a schematic representation of PDP-induced translocation of calreticulin (CRT) from the endoplasmic reticulum to the cell surface as a marker of immunogenic cell death.

FIG. 9B shows an increase in the surface exposure of calreticulin in CT1BA5 cells is light dose dependent following incubation with iTPALs (17α).

FIG. 10A shows a schematic representation of the photomodulation of collagen hydrogels followed by imaging.

FIG. 10B shows representative SHG images of collagen hydrogels on the untreated control (left), with iTPALs (17α) incubation and no irradiation (middle), and with iTPALs (17α) incubation and 690 nm irradiation (right).

FIG. 10C shows collagen fiber area fraction untreated and following treatment with iTPALs (17α) and iTPALs (17α)+690 nm irradiation.

FIG. 10D shows collagen fiber diameter untreated and following treatment with iTPALs (17α) and iTPALs (17α)+690 nm irradiation.

FIG. 10E shows collagen fiber area fraction untreated and following treatment with iTPALs (17α)+690 nm irradiation and iTPALs (35α)+690 nm irradiation.

FIG. 11A shows a schematic representation of the photomodulation of collagen hydrogels followed by imaging.

FIG. 11B shows representative SHG images of collagen hydrogels on the untreated control (left), with iTPALs (35α) incubation and no irradiation (middle), and with iTPALs (35α) incubation and 690 nm irradiation (right).

FIG. 11C shows collagen fiber area fraction untreated and following treatment with iTPALs (35α) and iTPALs (35α)+690 nm irradiation.

FIG. 11D shows collagen fiber diameter untreated and following treatment with iTPALs (35α) and iTPALs (35α)+690 nm irradiation.

FIG. 12A shows a representation of the self-delivery of iTPALs (17α) through collagen that is potentiated by 690 nm irradiation.

FIG. 12B shows quantification of the penetration of iTPALs (17α) through the collagen hydrogel from the top where they were added to the hydrogel.

FIG. 12C shows a graph demonstrating accumulation of iTPALs (17α) into the collagen hydrogel following 690 nm irradiation.

FIG. 13A shows a representation of the self-delivery of iTPALs (35α) through collagen that is potentiated by 690 nm irradiation.

FIG. 13B shows quantification of the penetration of iTPALs (35α) through the collagen hydrogel from the top where they were added to the hydrogel.

FIG. 13C shows a graph demonstrating accumulation of iTPALs (35α) into the collagen hydrogel following 690 nm irradiation.

FIG. 13D shows a comparison of total collagen delivery for iTPALs (17α), iTPALs (17α)+690 nm irradiation, iTPALs (35α), and iTPALs (35α)+690 nm irradiation.

FIG. 14A shows a schematic representation of a timeline of an animal study conducted in C57BL/6 mice bearing CT1BA5 tumors.

FIG. 14B shows representative images of mice bearing CT1BA5 tumors, administered with iTPALs (17α) without (top) and with (bottom) PDP using 690 nm irradiation at 0.5 h following intravenous administration.

FIG. 14C shows quantification of longitudinal tumor fluorescence imaging depicting the delivery and retention of iTPALs (17α) with 0 h, 9 h PDP and without PDP.

FIG. 14D shows Area Under Curve (AUC) analyses showing the total accumulation of iTPALs (17α) into the tumor.

FIG. 15A shows a spline plot of the tumor volumes of mice treated with iTPALs (17α) and sham IgG-PALs and a single dose PDP (0 h) and the untreated control.

FIG. 15B shows area under curve of tumor volumes until day 16 in mice treated with a single dose PDP (0 h) using iTPALs (17α) in comparison to sham IgG-PALs (0 h PDP) and the untreated control.

FIG. 15C shows a spline plot of the tumor volumes of mice treated with iTPALs (17α) and either no PDP, a single dose of PDP (0 h) and 9 h PDP.

FIG. 15D shows area under curve of tumor volumes in mice treated with a single dose of PDP (0 h) using iTPALs (17α) in comparison to iTPALs (17α) with 9 h PDP, iTPALs (17α) without PDP, and the untreated control.

FIG. 15E shows Kaplan-Meier plots representing the probability of progression free survival (tumor volume <500 m³) in mice treated with a single dose of PDP (0 h) using iTPALs (17α) in comparison to iTPALs (17α) with 9 h PDP, sham IgG-PALs without PDP, and the untreated control.

FIG. 15F shows Kaplan-Meier plots representing the probability of overall survival mice treated with a single dose of PDP (0 h) using iTPALs (17α) in comparison to iTPALs (17α) with 9 h PDP, sham IgG-PALs without PDP, and the untreated control.

FIG. 16A tumor growth in tumors were treated with 0 h PDP using iTPALs (17α) in comparison to 0 h PDP using sham IgG-PALs.

FIG. 16B shows tumor growth curves in mice treated with PDP using IgG-PALs at different drug light interval (0 h, 9 h) until day 30.

FIG. 16C shows immunofluorescence staining of CT1BA5 tumors 7 days after 0 h PDP using the iTPALs (17α).

FIG. 17A shows tumor uptake of iTPALs and overall survival following PDP.

FIG. 17B shows tumor uptake of sham IgG-PALs and overall survival following PDP.

FIG. 18A shows representative immunofluorescence images depicting decreased fibroblast contents on the cyrosectioned CT1BA5 tumor tissues when treated with iTPALs (17α)+PDP.

FIG. 18B shows quantitative analysis of fluorescence specific to fibroblasts from 10 ROIs spanning the entire tumor cross-section from 6 tumor samples for each condition.

FIG. 18C shows representative second harmonic generation (SHG) images of cryosectioned CT1BA5 tumor tissues showing reduced collagen area fraction when treated with iTPALs (17α)+PDP.

FIG. 18D shows quantitative collagen area fraction on the control tumor tissues and iTPALs (17α)+PDP treated tumor tissues depicting reduction in collagen fiber area fraction with the treatment.

FIG. 19 shows PDAC tumor infiltration of stem-like CD8+ T cells.

FIG. 20 shows synergistic results when combining light activation of iTPALs with nanoliposomal irinotecan.

FIG. 21A shows cellular uptake of LNP BPD-PC and Lipo BPD-PC at 24 h in CT1BA5 cells.

FIG. 21B shows expression of immunogenic cell death marker calreticulin in CT1BA5 cells following no PDT and PDT with LNP BPD-PC and Lipo BPD-PC with 20 J/cm² fluence.

### DETAILED DESCRIPTION OF PREFERRED EMBODIMENTS

The present disclosure relates to photoactivatable nanoparticles having attached immune checkpoint targeting and blocking features that allow for effective self-delivery and improved outcomes.

In preferred embodiments, the photoactivatable nanoparticles can be liposomes, solid lipid nanoparticles, organic or inorganic nanoparticles, self-assembled nanoparticles, crystalline nanoparticles, amorphous nanoparticles, lipid micelles, polymer micelles, hybrid micelles, or any other lipid or non-lipid based nanoassembly. The light activatable features can be BPD-PC activated by red or blue light, or any suitable light activatable feature, including but not limited to near-infrared light. The immune checkpoint targeting feature can target PD-L1 or other suitable immune checkpoints, including but not limited to PD-L2, CD80, CD86, HVEM, GAL-9, MHCII, CD40, TNFR, CD155, MCH, or others. The photoactivatable nanoparticles can contain varying densities of immune checkpoint targeting features.

In certain preferred embodiments, the photoactivatable nanoparticles are PD-L1 immune checkpoint targeted photoactivable liposomes (iTPALs). In additional preferred embodiments, the iTPALs have a varying number of α-PD-L1 antibodies attached on the surface, such as between 1 and 100 α-PD-L1 antibodies, or between 1 and 35 α-PD-L1 antibodies. In further preferred embodiments, the iTPALs have seventeen (17) α-PD-L1 antibodies attached on the surface and are denoted iTPALs (17α).

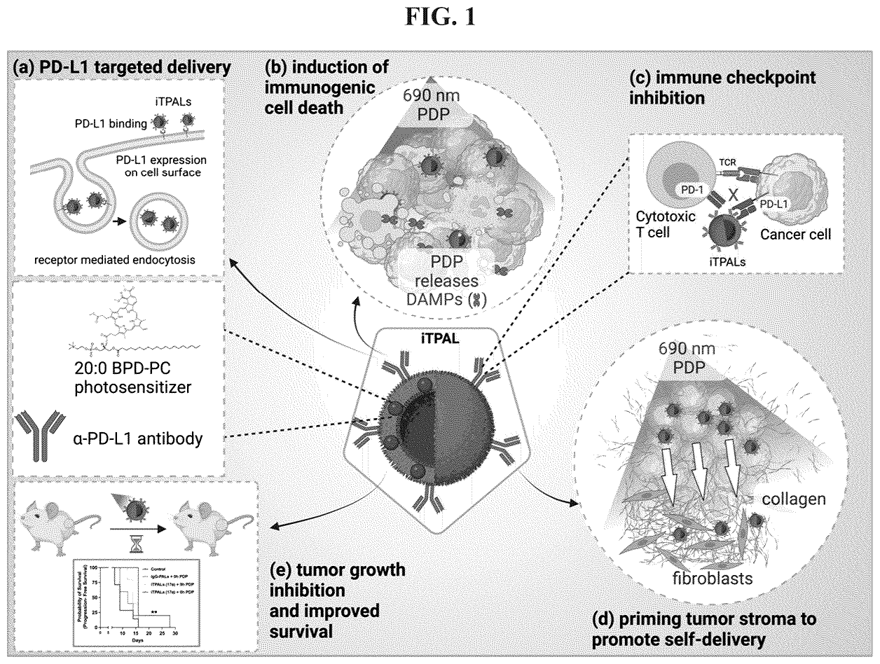

FIG. 1 shows a schematic representation of the utility of PD-L1 immune checkpoint targeted photoactivable liposomes (iTPALs (17α)) according to preferred embodiments described herein. The preferred embodiments of iTPALs (17α) contain a lipidated BPD-PC PS variant in the hydrophobic bilayer and α-PD-L1 antibodies on the surface. These iTPALs (17α) have following salient features: (1) facilitate tumor cell targeted delivery through PD-L1 receptor-mediated endocytosis, (2) induce immunogenic cell death upon light activation, which is characterized by the release of Damage Associated Molecular Patterns (DAMPs e.g. calreticulin), (3) block the PD-1/PD-L1 immune checkpoint between cytotoxic T cells and cancer cells, (4) remediate desmoplasia by photomodulating tumor collagen to promote the self-delivery through PDAC tumors, and (5) inhibit tumor growth and improve survival even with a single priming dose.

Preferred embodiments described herein related to photoactivable liposomes for simultaneous PD-L1 immune checkpoint targeting and blocking (iTPALs), which are capable of modulating the PDAC tumor stroma and promoting self-delivery upon 690 nm light activation. In doing so, iTPALs provide significant tumor growth inhibition and prolonged survival in an aggressive model of PDAC with only a single priming dose. The iTPALs described herein specifically target, bind, and internalize into cancer cells, which in turn, improves cancer cell phototoxicity. The capability of the iTPALs to efficiently block the PD-L1/PD-1 immune checkpoint at a dose where the free α-PD-L1 antibodies are ineffective is a significant innovative feature, and one that holds potential for lowering immune-related toxicity of immunotherapy in patients. Additionally, 690 nm light activation of iTPALs disrupts tumor collagen, reduces tumor fibroblasts, and promotes their self-delivery in vitro and in vivo which emphasizes the significance of iTPALs to overcome the most significant barrier in nanoparticle delivery in PDAC tumors. Interestingly, self-delivery of iTPALs directly correlated with overall survival in mice. PDP-enhanced combination chemo-immunotherapy may be useful in treating desmoplastic PDAC, which is in critical need of transformative innovations to prolong patient survival.

Accordingly, preferred embodiments disclosed herein relate to photoactivatable nanoparticles capable of self-delivery for use as therapeutic agents in immunotherapy treatments. The photoactivatable nanoparticles are made up of nanoparticles, at least one photosensitizer attached to the nanoparticles, and at least one immune checkpoint targeting feature attached to the nanoparticles. The immune checkpoint targeting feature inhibits an immune checkpoint in a tumor or cancer cell. The photoactivatable nanoparticles are self-delivered to the tumor or cancer cell through light activation of the photosensitizer.

In additional preferred embodiments, the nanoparticles of the photoactivatable nanoparticles are liposomes, solid lipid nanoparticles, organic or inorganic nanoparticles, self-assembled nanoparticles, crystalline nanoparticles, amorphous nanoparticles, lipid micelles, polymer micelles, hybrid micelles, or combinations thereof. The photosensitizer may be BPD-PC, as well as other suitable photosensitizers, including but not limited to porphyrins, phthalocyanines, chlorins, bacteriochlorins, phenothiazinium dyes, and derivatives thereof. The immune checkpoint targeting feature may be an α-PD-L1 antibody. Up to 35 immune checkpoint targeting features may be attached to the nanoparticle. In preferred embodiments, seventeen (17) α-PD-L1 antibodies are attached to the nanoparticle.

Additional preferred embodiments include a pharmaceutical composition for use in inhibiting tumor growth in a patient comprising a therapeutically effective amount of the photoactivatable nanoparticles of claim 1 and a pharmaceutically acceptable excipient, adjuvant, carrier, buffer, stabilizer, or mixture thereof. The pharmaceutical composition may also include therapeutically effective amounts of one or more additional treatment agents, wherein the one or more additional treatment agents are chemotherapy agents, small molecular weight inhibitors, targeted drugs, nucleic acids, mRNA, DNA, radiosensitizers, or combinations thereof.

A “therapeutically effective amount” is to be understood as an amount of an exemplary pharmaceutical composition comprising photoactivatable nanoparticles according to preferred embodiments described herein that is sufficient to reduce or inhibit tumor growth in a patient. The actual amount, rate and time-course of administration will depend on the nature and severity of the tumor being treated. Prescription of treatment is within the responsibility of general practitioners and other medical doctors. The pharmaceutically acceptable excipient, adjuvant, carrier, buffer or stabilizer should be non-toxic and should not interfere with the efficacy of the active ingredient. The precise nature of the carrier or other material will depend on the route of administration, which may be oral, or by injection, such as cutaneous, subcutaneous, or intravenous injection, or by dry powder inhaler.

Additional preferred embodiments described herein relate to a method for inhibiting tumor growth in a patient having a tumor by administering a pharmaceutical composition to the patient, wherein the pharmaceutical composition comprises photoactivatable nanoparticles according to preferred embodiments disclosed herein and applying light activation to a region of the patient in proximity to the tumor, whereby the photoactivatable nanoparticles are self-delivered to the tumor, and whereby the photoactivatable nanoparticles inhibit growth of the tumor. In preferred embodiments, the light activation is applied at 690 nm. In preferred embodiments, the method may also include the step of administering one or more additional treatments for inhibiting tumor growth to the patient in addition to the photoactivatable nanoparticles. The one or more additional treatments may be chemotherapy, small molecular weight inhibitors, targeted drugs, nucleic acids, mRNA, DNA, radiosensitizers, or combinations thereof.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin. For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as sodium chloride solution, Ringer's solution, or lactated Ringer's solution. Preservatives, stabilizers, buffers, antioxidants and/or other additives may be included as required.

In another aspect, there is provided the use in the manufacture of a medicament a therapeutically effective amount of a pharmaceutical composition comprising photoactivatable nanoparticles as defined above for administration to a subject.

The term “therapeutically effective amount” means a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

Further aspects of the present invention will become apparent from the following description given by way of example only.

### EXAMPLES

### Experimental Procedures

Preparation and characterization of liposomal benzoporphyrin derivate (Lipo-BPD) and liposomal lipid-anchored benzoporphyrin derivate (Liposomal BPD-PC): Benzoporphyrin derivative (BPD; US Pharmacopeia) was conjugated to 1-arachidoyl-2-hydroxy-sn-glycero-3-phosphocholine (20:0 Lyso PC; Avanti) to yield lipid-anchored benzoporphyrin derivate (BPD-PC), according to our previously published methods[1-3]. Briefly, BPD, 4-(dimethylamino) pyridine (DMAP; Sigma-Aldrich), 1-ethyl-3-(3-dimethylaminopropyl) carbodiimide (EDC; Sigma-Aldrich), N, N-diisopropylethylamine (DIPEA; Sigma-Aldrich), and 20:0 Lyso PC were mixed at molar ratios of 1:5:50:25:60, respectively, in 5 ml of dichloromethane (DCM; Fisher Scientific, high-performance liquid chromatography [HPLC] grade) and stirred at 2500 RPM for 72 h at room temperature. BPD-PC was purified using preparatory thin-layer chromatography and extracted in a 2:1 DCM/methanol mixture. The extracted BPD-PC was finally filtered through a 0.22 μm polytetrafluoroethylene (PTFE) filter and stored in chloroform at −20° C. in the dark. For the synthesis of liposomal BPD-PC, the lipids 1,2-dipalmitoyl-sn-glycero-3-phosphocholine (DPPC; Avanti), dioleoyl phosphatidylglycerol (DOPG; Avanti), 1,2-distearoyl-sn-glycero-3-phosphoethanolamine with conjugated methoxyl poly(ethylene glycol) (DSPE-mPEG₂₀₀₀; Avanti), 1,2-distearoyl-sn-glycero-3-phosphoethanolamine conjugated polyethylene glycol with dibenzocyclooctyne (DSPE-PEG₂₀₀₀-DBCO; Avanti), and 20:0 Lyso-PC-BPD in chloroform were mixed at a molar ratio of 0.869:0.077:0.044:0.005:0.006, respectively. Similarly, for Lipo-BPD, the lipids DPPC, DOPG, DSPE-mPEG₂₀₀₀, DSPE-mPEG₂₀₀₀-DBCO, and BPD in chloroform were mixed at the same molar ratios as described above. Both liposomes were prepared using a conventional thin-film hydration method by hydrating in Dulbecco's Phosphate Buffered Saline (DPBS; no Calcium, no Magnesium; Corning) followed by ultrasonication (Ultrasonic probe sonicator; Fisher Scientific) for a total of 30 mins (20 s on/40 s off cycles) at 42° C. in the dark. UV-Visible spectrophotometry was used to measure the concentration of BPD and BPD-PC in the liposomes using ε687 nm=34,895/M/cm in dimethyl sulfoxide (DMSO; Sigma-Aldrich).

Conjugation and characterization of α-PD-L1 antibodies modified with Alexa Fluor 488-NHS and NHS-PEG₄-Azide: Rat anti-mouse α-PD-L1 antibody (B7-H1; BioXCell) was diluted to 2 mg/ml in DPBS, Alexa Fluor 488-N-hydroxysuccinimide ester (AF488-NHS; Fisher Scientific) was diluted to 1 mg/ml in DMSO and NHS-PEG₄-Azide (NHS-PEG₄-N₃; Fisher Scientific) was diluted to 5 mg/ml in DMSO. 500 μl of 2 mg/ml α-PD-L1 antibodies were then mixed with AF488-NHS and NHS-PEG₄-N₃ at quantities corresponding to a 2.5-fold molar excess of each molecule to α-PD-L1 for 24 h at room temperature by orbital rotation. Unreacted AF488-NHS and NHS-PEG₄-N₃ were removed by size exclusion chromatography using Sephadex G25-M PD-10 columns (Fisher Scientific) pre-equilibrated with DPBS. The purified α-PD-L1 antibodies conjugated to both AF488 and PEG₄-N₃ (α-PD-L1-PEG₄-N₃) were collected and concentrated by centrifugation in 30 kDa ultrafiltration tubes (EMD Millipore) at 2,500×g for 20 mins at 4° C. UV-Visible spectrophotometry was used to measure the concentrations of α-PD-L1 and AF488 using ε280 nm=210,000/M/cm and ε494 nm=71,000/M/cm in DPBS respectively with a correction factor of 0.11 for AF488 at 280 nm. Similarly, IgG isotype control (2 mg/ml; Fisher Scientific) was also conjugated with AF488-NHS and NHS-PEG₄-N₃, and purified to obtain IgG-PEG₄-N₃ using the same procedures as described for α-PD-L1.

Conjugation of α-PD-L1-PEG₄-N₃ to liposomes and characterization: α-PD-L1-PEG₄-N₃ was reacted with liposomal BPD-PC at varying molar ratios of liposomes-to-antibodies, (1:0, 1:10, 1:25, 1:50, 1:75, 1:100, and 1:200) for 24 h at room temperature with orbital rotation. The excess unbound antibodies were separated using size exclusion chromatography columns packed with Sepharose CL-4B columns (Sigma-Aldrich) equilibrated with DPBS. The concentration of BPD-PC equivalent in the α-PD-L1 conjugated liposomes (iTPALs) was determined by UV-Visible spectrophotometry using ε687 nm=34,895/M/cm in DMSO. The conjugation efficiency of α-PD-L1-PEG₄-N₃ with liposomal BPD-PC was calculated from standard curves of α-PD-L1-PEG₄-N₃ in DPBS generated using fluorometry (Exc_{480 nm}/Emi_{517 nm}). Untargeted liposomal-BPD-PC was also passed through the Sepharose CL-4B columns (Sigma-Aldrich) and were referred to as photoactivable liposomes (PALs). Fluorescence emission values of iTPALs were normalized to the concentration of PALs within each preparation. The hydrodynamic diameters, polydispersity indices (PDI), and ζ-potentials of all the prepared liposomes were measured using Dynamic Light Scattering (DLS; Zetasizer Pro, Malvern Instruments, Malvern, UK). For hydrodynamic diameter and PDI measurements, 2 l of the liposomes were diluted in 1 ml of DPBS and measured using the Zetasizer Pro in triplicate. Similarly, for ζ-potential measurements, 10 μl of the liposomes were diluted in 1 ml of 0.9% w/v NaCl in H₂O and measured in triplicate using the Zetasizer Pro. Similarly, IgG-PEG₄-N₃ was also conjugated with liposomal BPD-PC and characterized using the same procedure as described above to yield sham IgG-PAL constructs that have no molecular specificity.

Cell Culture: AT84 (murine head and neck cancer) cells were a kind gift from Dr. Michael Story, University of Texas Southwestern Medical Center (UTSW), and were cultured in Roswell Park Memorial Institute 1640 media (RPMI 1640; Sigma-Aldrich) containing 10% Fetal Bovine Serum (FBS; R&D Systems), 1% L-glutamine (Sigma-Aldrich) and 1× penicillin streptomycin (Corning). LLC (murine Lewis lung carcinoma) cells were cultured in RPMI 1640 media containing 10% FBS and 1× penicillin streptomycin. MC38 (murine colorectal cancer) cells were cultured in Dulbecco's Modified Eagle's Medium (DMEM) F12 Ham (Sigma-Aldrich) with 5% FBS, 1% L-glutamine, and 1× penicillin streptomycin. ID8 (murine ovarian cancer) cells were purchased from Sigma and were maintained in DMEM with 4% Embryonic Stem cell (ES) qualified FBS, 1× insulin serum (Sigma-Aldrich) and 1× penicillin streptomycin. CT1BA5 and BMFA3 cells are isogenic pancreatic cancer cell lines derived from KPfC (Kras^{LSL-G12D}; Trp53^{fl/fl}; PDX^{Cre/+}) mice. Both cell lines were cultured in DMEM with 10% FBS and 1× penicillin streptomycin. All cells were maintained at 37° C. in a humidified incubator (Fisher Scientific) under 5% CO2. To harvest the cells, they were first washed with 1× DPBS, detached with 0.05% Trypsin (Corning), and collected in their respective media.

Cellular uptake studies: Cellular uptake studies were performed on CT1BA5 cells using flow cytometry. The liposomes were conjugated to a 0, 10, 25, 50, 75, 100, and 200-fold molar excess of antibodies to a liposome, as described earlier. The conjugated liposomes were purified and named based on the actual number of antibodies conjugated to the surface of the liposomes. Since 0, 10, 25, 50, 75, 100 and 200 antibodies that were reacted per liposome yielded 0, 3, 9, 17, 27, 35 and 37 antibodies conjugated per liposome, they were named PALs, iTPALs (3α), iTPALs (9α), iTPALs (17α), iTPALs (27α), iTPALs (35α) and iTPALs (37α), respectively.

Confluent CT1BA5 cells were first washed in DPBS, trypsinized, and collected in their respective media. 10,000 CT1BA5 cells/well were seeded in 96 well plates (Corning) for 24 h with 40 ng/ml of mouse Interferon Gamma Protein (mIFN-γ; Sino Biological). The cells were then incubated at 37° C. in the dark with 250 nM (BPD-PC equivalent) of the prepared liposomes for 1 h, 6 h, 9 h, 24 h, and 48 h. After each time point, the cells were trypsinized and resuspended in 300 μl of Fluorescence-Activated Cell Sorting (FACS) buffer (1% FBS solution in DPBS) and transferred into the flow cytometry tubes. They were then analyzed for BPD fluorescence using a Flow cytometry analyzer (BD Fortessa) with a 405 nm laser and a 710/50 nm bandpass detector.

Stability of iTPALs: The stability of the iTPALs (17α) was evaluated at two different temperatures, 4° C. (in DPBS) and 37° C. (in serum-containing media) over 8 days. To determine their shelf life at 4° C., 10 M of the iTPALs (17α) were incubated in DPBS. For serum at 37° C., 10 μM of the iTPALs (17α) were incubated in DMEM with 10% FBS. Following incubation, the hydrodynamic diameters and PDIs were measured daily to assess any changes in size and polydispersity using DLS, as described above.

Photobleaching of iTPALs: To assess the photobleaching of the Lipo-BPD, PALs, and iTPALs (17α) constructs, they were diluted to 40 μM BPD equivalent in DPBS. 100 μl aliquots of each sample were placed in triplicates into a transparent bottom white wall 96 well plate (Corning). The plate was then irradiated with 690 nm light at an irradiance of 27 mW/cm² and fluences of 0, 5, 15, 30, and 50 J/cm². The fluorescence emission of BPD was then measured after each irradiation cycle using an excitation wavelength of 430 nm and an emission wavelength of 690 nm using a Tecan Spark Plate Reader. Photobleaching was monitored as a function of decreasing BPD fluorescence with increasing light fluence.

Reactive Molecular Species (RMS) generation: Singlet oxygen generation by Lipo-BPD, PALs and iTPALs (17α) was measured using the colorimetric probe anthracene-9, 10-dipropionic acid (ADPA; Fisher Scientific) and the fluorometric probe singlet oxygen sensor green (SOSG; Fisher Scientific). Hydroxyl radical generation and peroxynitrite generation were measured using the fluorometric probe hydroxyphenyl fluorescein (HPF; Fisher Scientific). 5 μl of 6 mM ADPA in ethanol was added to 100 μl of 5 μM BPD-PC equivalent of Lipo-BPD, PALs and iTPALs in DPBS in a white wall, transparent bottom 96 well plate (Corning). The absorbance after each irradiation from 0.5 J/cm² to 3 J/cm² with 690 nm light (BioLambda) at 25 mW/cm² irradiance was measured at 378 nm using a Tecan Spark Plate Reader. Similarly, 10 μl of 50 mM SOSG was added to 100 μl of 5 μM BPD-PC equivalent of Lipo-BPD, PALs and iTPALs in DPBS in a white wall, transparent bottom 96 well plate (Corning). The fluorescence emission was then measured at 530 nm using an excitation wavelength of 460 nm using a Tecan Spark Plate Reader during irradiation with 690 nm light at 25 mW/cm² from 0.5 J/cm² to 3 J/cm². To measure hydroxyl radical and peroxynitrite generation, 20 μl of 200 mM HPF were added to 100 μl of the 5 μM BPD-PC equivalent of Lipo-BPD, PALs and iTPALs in DPBS in a white wall, transparent bottom 96 well plate (Corning). The fluorescence emission was then measured at 530 nm using an excitation wavelength of 460 nm using a Tecan Spark Plate Reader during irradiation at 690 nm at 25 mW/cm² from 0.5 J/cm² to 2.5 J/cm².

Fold increase in PD-L1 expression and binding specificity of iTPALs: Confluent AT84, LLC, MC38, ID8, CT1BA5 and BMFA3 cells were washed in PBS, trypsinized, and collected in their respective media. The cells were counted and diluted to seed 1.5×10⁵ cells per well in a 6 well plate (Corning) overnight. The next day, the media was replaced with fresh media containing 40 ng/ml of mIFN-γ and incubated for an additional 24 h. The cells were then trypsinized, dispersed in their respective media and counted using a cell counter (Bio-Rad). Cells were diluted in their respective media to obtain a concentration of 5×10⁵ cells/ml. Then, 100 μl of the counted cells were placed in the 1.5 ml microcentrifuge tubes. To calculate the fold increase in PD-L1 expression, 100 μl of 40 g/ml α-PD-L1 equivalent of α-PD-L1-AF488 were then added to the cells whereas to monitor the binding specificity of iTPALs (17α), 100 μl of 500 nM BPD-PC equivalent of either 100 μl PALs or TPALs (17α) added to the cells and incubated at 37° C. for 30 mins in the dark. The cells were then pelleted by ultracentrifugation for 5 mins at 1000×g, and the supernatants containing free unbound PALs and iTPALs (17α) were discarded. The cells were then resuspended in 300 μl of FACS buffer and transferred into flow cytometry tubes. For each condition, 10,000 cells were analyzed using a BD Fortessa flow cytometry analyzer with a 405 nm laser/710 nm bandpass detector pair for BPD-PC fluorescence and a 488 nm laser/530 nm detector pair for AF488 fluorescence. The data was then analyzed using FlowJo to calculate the median fluorescence of BPD-PC and AF488. The increase in PD-L1 expression after treating the cells with mIFN-γ was calculated as shown in Equation 1 below and the binding specificity of iTPALs (17α) was calculated as shown in Equation 2 below.

$$\begin{matrix}Formula to calculate the fold increase in PD - L 1 expression following the treatment of cells with mIFN - γ . \\ Fold increase in PD - L 1 expression with mIFN - γ = \frac{Mean Intensity of α - PD - L 1 in cells treated with mIFN - γ}{Mean Intensity of α - PD - L 1 in intreated cells}\end{matrix} \qquad Equation 1$$

$$\begin{matrix}Formula to calculate the binding specificity of iTPALs ( 17 α ) . \\ Binding specificity of iTPALs ( 17 α ) = \frac{Mean Intensity of BPD - PC in cells treated with iTPALs ( 17 α )}{Mean Intensity of BPD - PC in cells treated with PALs}\end{matrix} \qquad Equation 2$$

Additionally, the change in PD-L1 expression following PDP was measured in CT1BA5 cells. For this, the cells were first counted and diluted to seed 1.5×10⁵ cells per well in a 6 well plate (Corning) overnight. The next day, the media was replaced with fresh media containing 40 ng/ml of mIFN-γ and incubated for an additional 24 h. The media in the cells were then replaced with IC₂₀ value (25.2 nM) of iTPALs (17α) as generated from the molecular targeted phototoxicity experiment. Following 9 h incubation, each well was washed three times with media and irradiated with 40 J/cm² fluence and 25 mW/cm² irradiance of 690 nm light. After 24 h incubation, the cells were trypsinized, dispersed in their respective media and counted using a cell counter (Bio-Rad). Cells were diluted in their respective media to obtain a concentration of 5×10⁵ cells/ml. Then, 100 μl of the counted cells were placed in the 1.5 ml microcentrifuge tubes. 40 μg/ml α-PD-L1 equivalent of α-PD-L1-IRDye800 were then added to the cells and incubated at 37° C. for 30 mins in the dark. The cells were then pelleted by ultracentrifugation for 5 mins at 1000×g, and the supernatants containing free unbound α-PD-L1-IRDye800 were discarded. The cells were then resuspended in 300 μl of FACS buffer and transferred into flow cytometry tubes. For each condition, 10,000 cells were analyzed using a BD Fortessa flow cytometry analyzer with a 405 nm laser/710 nm bandpass detector pair for IRDye800 fluorescence The data was then analyzed using FlowJo to calculate the median fluorescence of IRDye800 and plotted.

Subcellular localization of iTPALs: 50,000 CT1BA5 cells with 40 ng/ml of mIFN-γ were seeded per well in a 96 well glass bottom, black wall plate (Cellvis). The next day, 2 μM BPD-PC equivalents of PALs and iTPALs (17α) containing media were added to the cells and incubated at 37° C. for 24 h in the dark. After incubation, media containing either 1 μg/ml of Hoechst (nuclei tracker; Cell Signaling Technology) or 50 nM of the Lysotracker (Cell Signaling Technology) were added to the wells and incubated at 37° C. in the dark for 1 h. The samples were then imaged for colocalization using an Olympus FV3000RS Confocal Laser Scanning Microscope at a 100× oil immersion objective with a 488 nm laser for Lysotracker excitation, a 405 nm laser for Hoechst excitation, and a 647 nm laser for iTPAL excitation.

Molecular targeted phototoxicity (MTT assay): Confluent AT84, LLC, MC38, ID8, CT1BA5 and BMFA3 cells were washed in PBS, trypsinized and collected in their respective media. They were then counted and diluted to 15,000 cells/ml after which 100 μl of the cells with 40 ng/ml of mIFN-γ were seeded per well in 96 well plates (Fisher Scientific). The next day, the cells were treated in 4 replicates with different concentrations of PALs and iTPALs (17α) ranging from 0.125 nM to 10,000 nM BPD-PC equivalent concentrations in media. After 9 h incubation, each well was washed three times with media and irradiated with 40 J/cm² fluence and 25 mW/cm² irradiance of 690 nm light. After 48 h incubation, 0.3 mg/ml of 3-(4,5-dimethylthiazol-2-yl)-2,5-diphenyltetrazolium bromide (MTT dye; Sigma-Aldrich) was added to each well and incubated for 2.5 h until the purple formazan crystals were formed. Media in each well was carefully replaced with 100 μl of DMSO and the absorbance was measured at 555 nm wavelength using a Tecan Spark Plate Reader. The absorbance in each well was corrected for the baseline absorbance of DMSO and the metabolic activity of the cells in each well was calculated.

Conjugation of PD-1 biotin with Streptavidin-Microspheres: The PD-1 biotin (PD-1 Protein, Mouse, Recombinant Biotinylated; Sino Biological) was incubated at 4° C. for 30 mins with streptavidin-conjugated microspheres (Streptavidin Fluoresbrite® YG Microspheres, 6.0 μm; Sino Biological), at a quantity of 15 g of biotinylated PD-1 per 1 mg of streptavidin-conjugated microspheres. The conjugated PD-1 microspheres were then washed three times in DPBS/BSA buffer (1% BSA in DPBS) by centrifugation at 10,000×g at room temperature to remove any excess unconjugated PD-1 biotin. Finally, the conjugated PD-1-coated microspheres were dispersed in DPBS/BSA buffer to obtain a final concentration of 10 mg/ml for further use.

Blocking PD-1/PD-L1 axis using iTPALs: 50,000 CT1BA5 or AT84 cells per well were seeded with 40 ng/ml of mIFN-γ in 96 well glass bottom, black wall plates (Cellvis). The next day, the cells were pre-blocked with 2.5 μpM (BPD-PC equivalent) of PALs, iTPALs and free α-PD-L1 antibodies (α-PD-L1 concentration equivalent to iTPALs; 7.5 nM) in triplicates for 1 h. The cells were then incubated with 20 μg/ml of PD-1-coated microspheres for 30 mins and then washed three times with media to remove unbound microspheres. The PD-1-coated microspheres in each well were then imaged using an Olympus FV3000RS Confocal Laser Scanning Microscope with a 20× objective and a 491 nm laser. Finally, the images were analyzed to obtain average numbers of PD-1 microspheres per ROI.

Immunogenic cell death: 50,000 CT1BA5 cells were seeded in a 6 well plate with 40 ng/ml of mIFN-γ and incubated for 24 h at 37° C. After 24 h, the media in each well was replaced with fresh media containing 250 nM BPD-PC equivalent of iTPALs (17α). Following 24 h incubation, the cells were washed three times with fresh media and were irradiated with 690 nm light at an irradiance of 25 mW/cm² and fluences of 25 J/cm², 50 J/cm², 100 J/cm² and incubated again for an additional 24 h. Cells were then washed with DPBS, trypsinized, collected in Eppendorf tubes, and rewashed twice with DPBS to remove any remaining trypsin or media. The cells in each Eppendorf tube were then fixed with 100 μl of 4% formaldehyde for 15 mins at room temperature. The cells were then washed with DPBS and resuspended in 100 μl of rabbit anti-mouse anti-calreticulin antibodies (1:400 dilution in antibody dilution buffer; Cell Signaling Technology; D3E6). After 1 h incubation, the cells were rewashed with DPBS and resuspended in 100 μl of goat anti-rabbit secondary antibodies conjugated with AF594 (1:500 dilution in antibody dilution buffer; Cell Signaling Technology; 8889S). The cells were then incubated at room temperature in the dark for 20 mins, washed with DPBS, and resuspended in FACS buffer. Finally, the cells were analyzed using a BD Fortessa flow cytometry analyzer with a 561 nm laser and a 610 nm bandpass detector. The data was then analyzed using FlowJo to calculate the median AF594 fluorescence of each sample.

Photomodulation of collagen hydrogels: Collagen hydrogels were formed by mixing acid-solubilized rat tail collagen I (Corning, Product No. 354249) with an equal volume of a neutralizing buffer (100 mM HEPES in 2× PBS, pH 7.3) while keeping both reagents on ice. Cold PBS was then added to achieve a final concentration of 4 mg/ml collagen. Aliquots of 200 μlwere transferred into cylindrical wells of polydimethylsiloxane (PDMS) with a diameter of 9 mm and a height of 3 mm. The collagen solution was allowed to polymerize inside a cell culture incubator (37° C., 5% CO₂) for 1 h. The resulting collagen hydrogels were then divided into three groups: the first group received no treatment (negative control), the second group received 5 μl of 10 μM iTPALs (35α) but was not exposed to light, while the third group received 5 μl of 10 μM iTPALs (35α) and after 1 h was irradiated using a 690 nm laser (Modulight) for PDP at an irradiance of 100 mW/cm² and a fluence of 100 J/cm². After equilibration, the gels were imaged using an Olympus MPE-RS TWIN Multiphoton Microscope equipped with a 25× water immersion objective lens (1.05 NA, 2 mm working distance). Upon excitation with 880 nm light, Second-Harmonic Generation (SHG) images were collected using a 410-460 nm bandpass filter to capture the fibrillar structure of the collagen network. Similarly, Two-Photon Fluorescence (TPF) intensity was collected using a 660-750 nm bandpass filter to quantify the fluorescence of iTPALs (35α). Image stacks were acquired starting at 100 μm above the top surface of the collagen gels and ending 1000 m deep into the gels, with step sizes of 10 μm between consecutive image planes. The mean signal intensity at any distance from the top surface of the gel was ascertained by averaging the signal intensity on the appropriate plane across three samples per experimental group. To characterize the structural organization of the collagen network, SHG images were acquired at a resolution of 1024×1024 pixels, with a pixel size of 0.124 μm/pixel, 2× line averaging, and 4× optical zoom. These images were post-processed using the image segmentation tool CT-FIRE to assess variations in mean collagen fiber length, thickness, and density across treatment groups.

In vivo delivery of iTPALs: All procedures were performed in accordance with Institutional Animal Care and Use Committee (IACUC) protocols (Protocol number: 19-10). Male C57BL/6 mice (20 g, 6 weeks old) purchased from Jackson Laboratory were maintained in a fully equipped animal facility in compliance with the NIH Guide for the Care and Use of Laboratory Animals at the University of Texas at Dallas Vivarium. A day prior to implantation, the hair on the left flank of mice were shaved with Wahl professional animal hair clippers. CT1BA5 cells were cultured in DMEM containing 10% FBS. When the cells reached 80% confluency, they were trypsinized and collected in media. The cells in the media were counted and washed three times with DPBS to remove any remaining media and trypsin. The cells were then diluted to obtain 1×106 cells in 20 μl sterile DPBS and implanted subcutaneously into the left flank of the mice. The tumor volumes were monitored after implantation using a digital vernier caliper. 16 d following tumor implantation, the tumors reached a volume of 300 mm³-500 mm³ as calculated using the formula ab2/2 (a=length of larger dimension, b=length of smaller dimension). On the treatment day, the mice were divided into two cohorts: iTPALs (17α) only and iTPALs (17α)+690 nm group with 5 mice on each arm. Before PDP, the mice were pre-emptively administered with subcutaneous Meloxicam SR analgesia to minimize any discomfort during PDP. The groups were then intravenously administered with 2 mg/kg of iTPALs (BPD-PC equivalent). Immediately following administration, tumors the PDP group were irradiated with a 690 nm laser at a fluence of 75 J/cm² and an irradiance of 100 mW/cm². The mice were then imaged using a LICOR PEARL Imaging System to measure the longitudinal tumor fluorescence signals arising from the tumor uptake of iTPALs (17α) at 0.5 h, 3 h, 6 h, 9 h, and 24 h, 48 h and 72 h. To quantify the fluorescence of iTPALs (17α) in the tumor, the Image Studio Work Area software was used to draw ROIs around the tumors and the skin fluorescence at distant regions was subtracted from the fluorescence of each tumor ROI. The data was then analyzed to assess the difference in the delivery of iTPALs (17α) into the tumors with and without PDP using GraphPad Prism v9.2.0.

In-vivo Photodynamic Priming. After 9 days of tumor implantation, the mice were randomly divided into 5 groups (Table 3, below). They were then administered with 1 mg/kg BPD-PC equivalent dose of either iTPALs (17α) or IgG-PAL sham control with similar number of antibodies. PDP with 690 nm of light was then given to the required group. Following the treatment, tumor volumes were measured in every alternate day until they die, or their volumes reach 2000 mm³ endpoint. The tumor volumes, probability of survival, area under curve (AUC) of tumor volume, progression-free survival and overall survival in mice were then plotted and analyzed using Graph Pad Prism v9.2.0. For progression-free survival analysis, 500 mm³ was used as a cut-off volume, whereas for overall survival, the day when a mouse was dead/euthanized was considered as an endpoint. Median survival was calculated automatically during overall survival analysis by Graph Pad Prism v9.2.0.

Immunofluorescence Analysis. After either 72 h or 8 d following PDP with iTPALs (17α), treated and untreated control mice were euthanized, and the tumors were harvested and cryopreserved in O.C.T (Tissue-Plus™ O.C.T. Compound; Fisher Scientific) for immunofluorescence. The immunofluorescence analysis was conducted to monitor the infiltration of CD8⁺ T cells into the tumor after 8 days of treatment with iTPALs (17α)+0 h PDP and monitor the effect of iTPALs (17α)+0 h PDP on tumor fibroblasts at 72 h following the treatment. For that, each OCT-embedded tumor was cryosectioned to obtain three 20 μm slices using the Cryostat-Leica CM1860. The slides were first thawed for 15 mins at room temperature, fixed in 1:1 Acetone: Methanol (Fisher Scientific) for 15 minutes at room temperature, air dried for 30 mins, washed with DPBS three times, and then blocked with Blocker BSA 1× (Fisher Scientific) for 30 mins. Next, the antibodies CD8 pre-conjugated with FITC (1:100 dilution; Cell Signaling Technology, Product number: 35467), TE-7 (1:100 dilution; Novus Biologicals, Product number: NBP2-50082) were diluted in antibody dilution buffer (Fisher Scientific). The diluted antibodies were then added to the slides and incubated at 4° C. overnight. The following day, the slides were washed three times with DPBS, and the secondary antibody, IgG fab2 AF555 (4409S; Cell Signaling Technology; 1:1000 dilution in antibody dilution buffer), added to the slides treated with TE-7 primary antibodies and incubated for two hours in the dark at room temperature. Subsequently, the secondary antibodies were washed three times with DPBS, and DAPI mounting media and cover slides were applied to the samples. Finally, nail polish was applied to the edges to prevent air from drying the slides.

The slides were then imaged with the Olympus VS120 Virtual Slide Microscope with a 20× objective. The fluorescence signals of FITC-labeled anti-CD8 antibodies (targeting CD8⁺ cytotoxic T cells) were obtained using excitation/emission wavelengths of 470 nm/525 nm. The fluorescence signals of AF 555 labeled secondary antibodies for TE-7 were obtained using 545 nm/605 nm. The fluorescence of the nuclear stain DAPI was visualized using excitation/emission wavelengths of 350 nm/460 nm. 10 random regions of interest (ROIs) spanning the entire tumor cross-section were analyzed from 6 tumor samples for each condition using Image J.

SHG Imaging of tumor collagen. The cryopreserved tissues from the mice treated with iTPALs (17α)+0 h PDP and euthanized at 72 h post treatment were used to monitor the photomodulation of tumor collagen. Each cryopreserved tumor was cryosectioned to obtain three 20 μm slices using the Cryostat-Leica CM1860. The slides were first thawed for 15 mins at room temperature and were fixed in 4% formaldehyde (Fisher Scientific) followed by three washes with DPBS. After washing, the sections were sealed with cover slides. Collagen imaging was performed using an Olympus MPE-RS TWIN Multiphoton Microscope using a tunable Mai Tai laser at 980 nm, a 25× water objective, and a 460-500 nm band pass emission filter. TIFF files of the SHG images of tumor tissue sections were subject to de-noising on ImageJ (2.0 pixel outliers using a threshold of 50), and a Li automatic threshold was applied to all images. Collagen fiber area fraction was then calculated from processed images using ImageJ.

### Development of Exemplary Nanoparticles

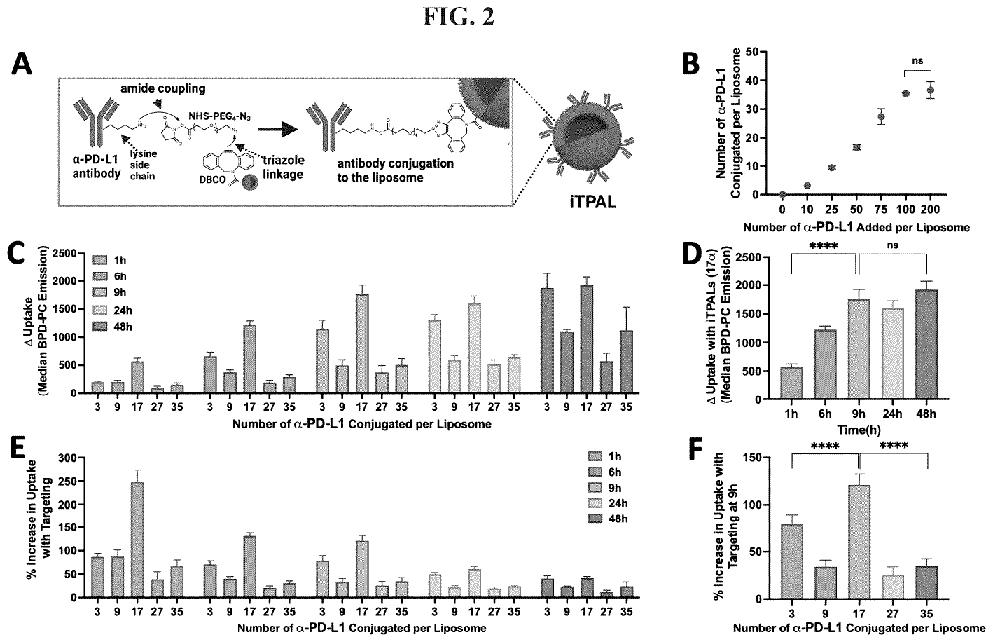

For an effective antibody targeted delivery of the liposomal PSs into the tumors, it is critical to first determine the stability of the system. Previous studies have shown that liposomal BPD is prone to premature leakage. Anchoring BPD with a lysophosholipid (BPD-PC) to the bilayer of liposomes promotes stability of the PS and prevents premature leakage. Therefore, a stable liposomal BPD-PC platform was first prepared for further conjugation with α-PD-L1 antibodies to prepare the exemplary PD-L1 immune checkpoint targeted photoactivable liposomes (iTPALs). FIG. 2A shows a schematic representation of the exemplary targeted photoactivable liposomes (iTPALs) entrapping the BPD-PC PS on the hydrophobic bilayer α-PD-L1 antibodies on the surface.

It is known that the ligand density on the surface of nanocarriers, such as liposomes, affects their pharmacokinetics, cellular binding, cellular internalization, and therapeutic efficacy. Liposomes were conjugated to varying densities of azido-PEG modified α-PD-L1 antibodies using copper-free click chemistry. Reacting 0, 10, 25, 50, 75, 100 and 200 α-PD-L1 antibodies per liposome resulted in 0, 3, 9, 17, 27, 35 and 37 α-PD-L1 antibodies bound per liposome, respectively. Liposomes without antibody conjugation are referred to as photoactivable liposomes (PALs). Liposomes conjugated to α-PD-L1 antibodies are referred to as iTPALs and the number of antibodies bound is denoted as (Xα), such as iTPALs (17α). FIG. 2B shows conjugation of varying α-PD-L1 antibody-to-liposome ratios on iTPALs. As shown in FIG. 2B, the number of α-PD-L1 antibodies conjugated increased linearly as more antibodies were reacted with the liposomes. Reacting 100 α-PD-L1 antibodies per liposome yielded iTPALs (35α). However, reacting 200 α-PD-L1 antibodies per liposome yielded iTPALs (37α) with no significant increase in the number of antibodies conjugated. As such, iTPALs (37α) synthesized by reacting 200 antibodies per liposome were excluded from the rest of the study. The plateau most likely suggests that there is a saturation of conjugation sites beyond ˜35 antibodies on the iTPAL surface. This is unexpected as a greater number of targeting ligands were previously conjugated to BPD-PC liposomes. Conjugation efficiencies may vary for α-PD-L1 antibodies.

To investigate how varying densities of α-PD-L1 antibodies affect the cellular internalization of iTPALs, uptake was quantified at different time points using flow cytometry and the fluorescence emission of BPD-PC. FIG. 2C shows median BPD emission from iTPALs obtained from the flow cytometry analysis on the cellular uptake of different α-PD-L1 antibody-to-liposome ratios (0, 3, 9, 17, 27, 35 antibodies per liposome) at 1 h, 6 h, 9 h, 24 h and 48 h of incubation in CT1BA5 cells. FIG. 2C shows the delta uptake (difference in uptake between iTPALs and untargeted PALs) in murine PDAC cells (CT1BA5). Among all densities of α-PD-L1 antibodies in iTPALs, iTPALs (17α) had the greatest delta uptake at all time points, which peaked at 9 h as shown in FIG. 2D. FIG. 2E shows percent improvement in cellular uptake of iTPALs with respect to untargeted PALs at different time points. Targeting improved the internalization of all densities of iTPALs with respect to untargeted PALs. However, the degree of improvement in internalization was not correlated with the density of antibodies per liposomal BPD-PC at all time points. Receptor-mediated internalization of liposomes is a complex process that balances multivalent binding that promotes receptor clustering and internalization, with steric effects that make receptor binding inefficient at higher antibody densities. FIG. 2F shows that iTPALs (17α) exhibited the higher percentage improvement in uptake and significantly improved the uptake of photosensitizing agent more than all other iTPAL variants. In FIG. 2, data are mean±S.D.; statistical significance was calculated on GraphPad Prism v9.2.0, ***: P<0.001, ****: P<0.0001.

These results were unexpected, as a previous study showed that 30 Cetuximab molecules per liposomal BPD-PC provided the highest cell specific internalization This suggests that the optimal density of antibodies for maximum cancer cell targeted internalization is dependent on the nature of the target receptor i.e., PD-L1 in this study, receptor clustering and how the receptors facilitate receptor mediated internalization. Not much is known about PD-L1 clustering and receptor mediated endocytosis. Overall, these results showed that iTPALs (17α) were optimal and were used for the majority of the remaining studies.

### Characterization of Nanoparticles

The hydrodynamic diameters, polydispersity index (PDI), and ζ-potentials of the developed iTPALs (17α) and untargeted PALs (n=5 independent formulations prepared) are summarized in Table 1 below.

TABLE 1

| | Hydrodynamic | Polydispersity | ζ-Potential | Conjugation |
|---|---|---|---|---|
| Sample | Diameter (nm) ^{[a]} | Index ^{[a]} | (mV) ^{[a]} | Efficiency ^{[a]} |
| PALs | 105.8 ± 2.7 | 0.11 ± 0.04 | −3.6 ± 1.8 | — |
| iTPALs | 117.5 ± 11.0 | 0.11 ± 0.05 | −3.4 ± 1.8 | 33.4% ± 1.7% |
| (17α) | | | | |

^{[a]} Mean ± S.D.

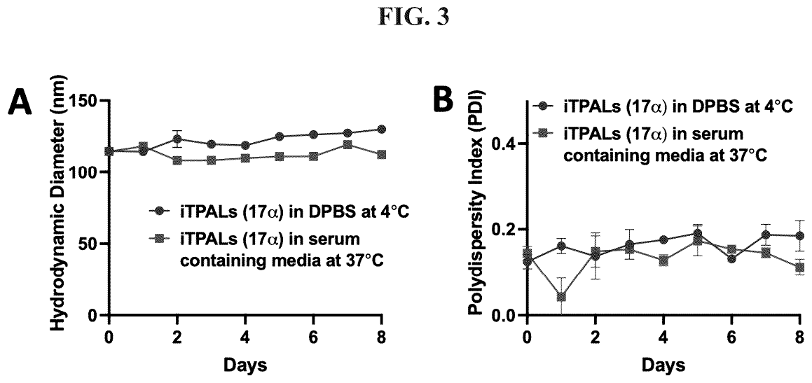

As shown in Table 1, the hydrodynamic diameter of liposomes increased (P<0.05) when α-PD-L1 antibodies were bound on their surface. The untargeted PALs were 105.8±2.7 nm in diameter, which increased to 117.5±11.0 nm in the iTPALs (17α) constructs. The polydispersity indices of PALs and iTPALs were all lower than 0.2, which suggests that the constructs are relatively monodisperse. Both the PALs and iTPALs exhibited a moderately anionic ζ-potential which is expected as 7.7 mol % of the anionic lipid DOPG was incorporated into the formulation. iTPALs (17α) had a conjugation efficiency of 33.4%±1.7%. Moreover, the shelf life and the stability of iTPALs (17α) were tested in DPBS at 4° C. and in serum-containing media at 37° C., respectively. FIG. 3 shows that iTPALs are physically stable during storage and incubation. FIG. 3A shows hydrodynamic diameter and FIG. 3B shows polydispersity indices of iTPALs (17α), which were unchanged during storage in DPBS at 4° C. and incubation in serum containing media at 37° C. for 8 days.

When liposomes come in contact with biological fluids, such as serum proteins, a protein corona forms that can increase their hydrodynamic diameters. However, in this study, iTPALs are conjugated with antibodies at the liposome surface, and the surface is not saturated by PD-L1 antibodies with only 17 antibodies per liposome. It is to be expected that the backfill of serum proteins onto the liposome would not significantly influence the hydrodynamic diameters of the iTPALs that contain antibodies at their surface. To further probe this, an experiment on liposomes that do not contain antibodies was conducted and the change in size was measured when incubated with serum containing media at 37° C. for 24 h. The size of these liposomes (PALs) increased significantly by 16 nm (p<0.0001), which is attributed to protein corona. So, while serum proteins are capable of adhering to these iTPALs, a change in hydrodynamic diameter is only detected when the liposomes contain no antibodies, which already increases their size by 12 nm.

### Reactive Molecular Species (Rms) Generation

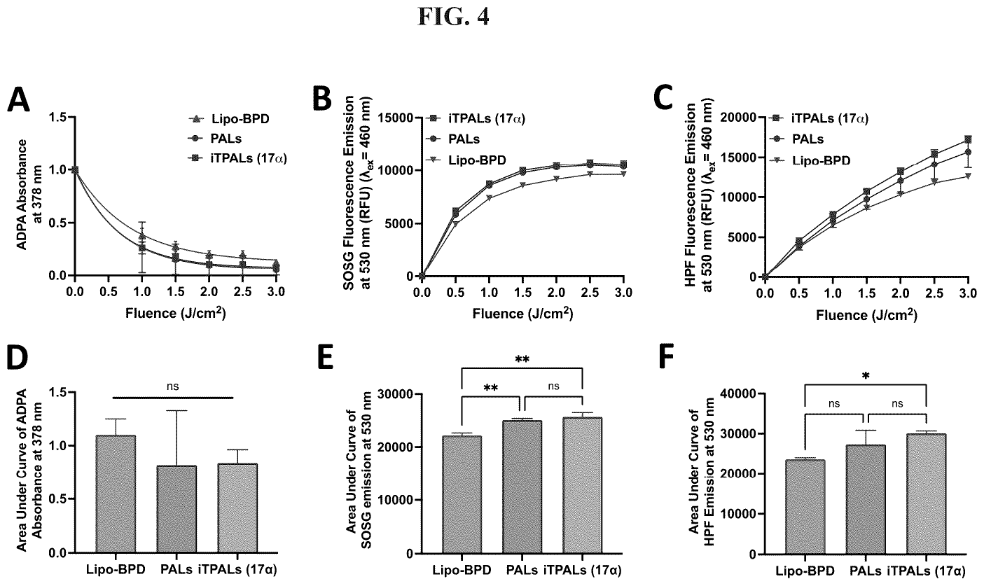

In preferred embodiments, iTPALs are developed to achieve enhanced cancer cell-targeted photokilling and PDP so it is critical that it maintains the RMS generating ability after conjugation of α-PD-L1 antibodies. The conjugation of Cetuximab, Holo-transferrin and Herceptin simultaneously to liposomal BPD-PC reduced RMS production by approximately 20%. This example explores whether the conjugation of 17 α-PD-L1 antibodies per iTPAL can impact RMS production. To monitor the RMS generation, anthracene-9, 10-dipropionic acid (ADPA), singlet oxygen sensor green (SOSG) and hydroxyphenyl fluorescein (HPF) probes were used where ADPA and SOSG measured the singlet oxygen generation and HPF measured other molecular species (hydroxyl radical and peroxynitrite anion) generation. Results shown in FIG. 4 demonstrate that PD-L1 antibody conjugation does not impair RMS photoproduction by iTPALs. Statistical significance was calculated using one-way ANOVA with a Tukey post-test on GraphPad Prism v9.2.0, *: P<0.1, **: P<0.01). FIGS. 4A and 4D show the generation of singlet oxygen as confirmed by the decreased absorbance of the colorimetric singlet oxygen probe ADPA with increasing fluence of 690 nm light. Results suggest that the singlet oxygen generation was comparable in Lipo-BPD, PALs and iTPALs (17α). FIGS. 4B and 4E show the generation of singlet oxygen as confirmed by the increased fluorescence of the fluorogenic singlet oxygen probe SOSG. Results suggest a significantly higher amount of RMS generation in PALs and iTPALs (17α) in comparison to Lipo-BPD whereas the difference between PALs and iTPALs (17α) was not significant. This might be because the BPD present in Lipo-BPD is unanchored and they stack together more easily to aggregate, self-degrade, and hinder the ROS generation. The discrepancy between the APDA data and SOSG data could be attributed to the markedly lower sensitivity of the colorimetric APDA probe.

FIGS. 4C and 4F show hydroxyl radical and peroxynitrite anion generation by the three formulations, as measured using the fluorogenic probe HPF, following 690 nm activation of iTPALs (17α). The differences are comparable to those of singlet oxygen generation, where the Lipo-BPD production of RMS is lower than the PALs and iTPALs (17α). Overall, these results suggest that the conjugation of α-PD-L1 antibodies on iTPALs (17α) does not impair the RMS generation.

### Photobleaching Study

Photobleaching, a decrease in luminescence emission as a result of photochemical activation, is common to all organic PSs. While a greater degree of photobleaching usually corresponds to a greater degree of therapeutic reactive molecular species (RMS) production, it also limits its activity, as a bleached PS is an ineffective one.

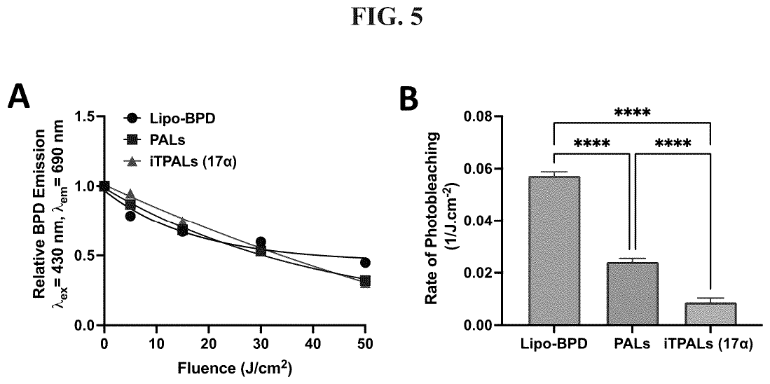

FIG. 5 shows that iTPALs are more photostable than other formulations. FIG. 5A shows decreasing fluorescence emission of Lipo-BPD, PALs and iTPALs (17α) in DPBS with increasing fluence of 690 nm irradiation. FIG. 5B shows photobleaching rate kinetics of Lipo-BPD, PALs and iTPALs (17α) demonstrating lower rates of photobleaching in iTPALs (17α) than in other formulations. Results were calculated by GraphPad Prism v9.2.0. Data are mean±S. D.; statistical significance was calculated on GraphPad Prism v9.2.0, ****: P<0.0001.

As shown in FIG. 5A, conventional Lipo-BPD photobleached the fastest following 690 nm irradiation, followed by untargeted PALs and iTPALs (17α). Previous studies showed liposomal BPD-PC photobleached faster than Lipo-BPD, although this could be attributed to higher BPD(-PC) to lipid ratios used in this study. Additionally, the rate of photobleaching of iTPALs (17α) was 84.7% lower than Lipo-BPD and 63.8% lower than PALs as shown in FIG. 5B. Although this could theoretically be attributed to the α-PD-L1 antibodies scavenging RMS, α-PD-L1 antibody conjugation does not compromise RMS generation, as discussed below. It is important to note that iTPALs (17α) are the most photostable and are therefore able to produce RMS for longer irradiation times.

### Binding Specificity of Itpals

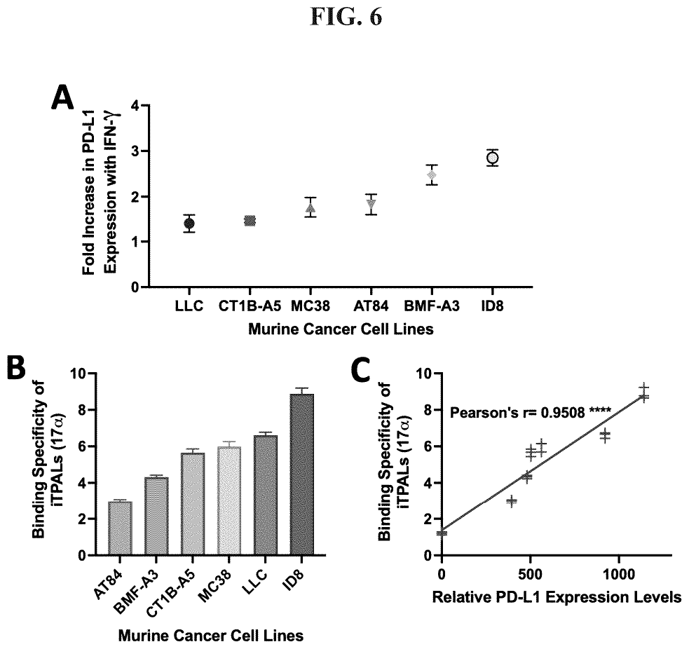

PD-L1 expression in tumors are regulated by cytokines, such as interferon gamma (IFN-γ) secreted by T cells. As such, studies typically use IFN-γ to induce expression of PD-L1 in cancer cells in vitro. Following incubation with mouse IFN-γ (mIFN-γ), the elevation of PD-L1 expression was first measured on six murine cancer cell lines. FIG. 6 shows that iTPALs (17α) bind specifically to PD-L1 expressing murine cancer cells. FIG. 6A shows that mIFN-γ increased the PD-L1 expression on murine cell lines: LLC (Lewis lung carcinoma), CT1BA5 (PDAC), MC38 (colorectal cancer), AT84 (head and neck cancer), BMFA3 (PDAC) and ID8 (ovarian cancer). FIG. 6B shows binding specificity of iTPALs (17α) to murine cancer cell lines, with respect to untargeted PALs. FIG. 6C shows PD-L1 expression in all cell lines tested correlates positively with the binding specificity of iTPALs (17α). (Data are mean±S.D; Correlation was analyzed using Pearson r correlations on GraphPad Prism v9.2.0, ****:P<0.0001)

mIFN-γ increased PD-L1 expression by 1.4-fold in LLC lung cancer cells, 1.5-fold in CT1BA5 PDAC cells, 1.8-fold in MC38 colon cancer cells, 1.8-fold in AT84 head and neck cancer cell, and 2.5-fold in BMFA3 PDAC cells and 2.9-fold in ID8 ovarian cancer cells. Using the optimal iTPALs (17α) construct, we measured the binding specificity to murine cancer cells with respect to the untargeted PALs. Following 30 min incubation, the binding specificity of iTPALs (17α) was found to range from 2.9-fold to 8.9-fold (FIG. 6B) with AT84 having the lowest specificity and ID8 having the highest specificity. Binding of iTPALs (17α) exhibited a positive linear correlation with cellular expression levels of PD-L1 (Pearson's r=0.9508, FIG. 6C). Although the iTPALs (35α) with higher α-PD-L1 antibodies conjugated exhibited 3-fold lower improvements in cellular uptake at 9 h than the optimal iTPALs (17α), binding specificity was in fact ˜2-fold higher with the iTPALs (35α). Binding of iTPALs (35α) also exhibited a positive linear correlation with cellular expression levels of PD-L1 (Pearson's r=9095). This interesting discrepancy between binding specificity and improvements in cellular uptake with targeting yet again speaks to the complexity of PD-L1 as a tumor targeting receptor. This contrasts with a previous study that demonstrates binding specificity towards EGFR is directly related to the improvements in cellular uptake with targeting. Because both binding specificity and improved cellular uptake of iTPALs are more important than the just binding specificity alone, the iTPALs (17α) construct is selected for all studies as it was efficient at both binding and internalization. Different wavelengths of light have been shown to alter the PD-L1 expression in vitro by up to 1.5-fold. In this study, we monitored the impact of 690 nm irradiation on PD-L1 expression in CT1BA5 cells in the presence and absence of iTPALs (17α). 40 J/cm² fluence of 690 nm light decreased the PD-L1 expression significantly by 28.1% and 22.2% in both the absence and presence of iTPALs (17α) respectively. These results also suggested that the decrease in PD-L1 expression with 690 nm light was not a result of the photodynamic process. Considering that this approach uses the light activation following cellular binding with iTPALs (17α), the mild decrease in PD-L1 expression following 690 nm light exposure is not anticipated to affect treatment response.

### Subcellular Localization of Itpals (17α)

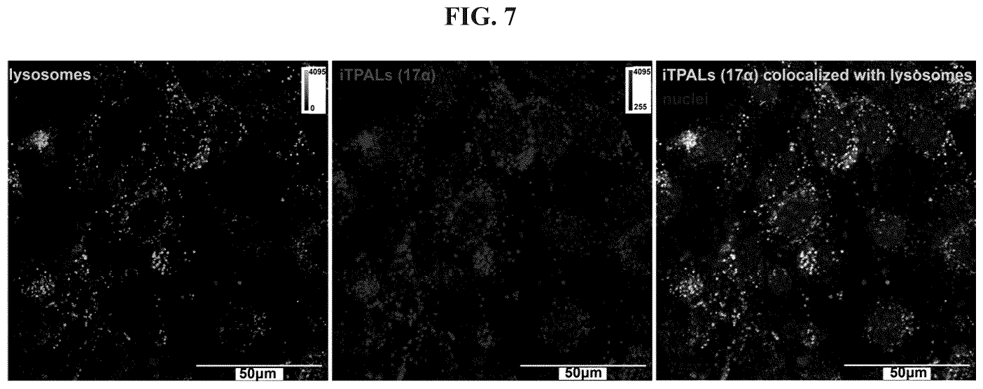

Targeted liposomes are known to internalize through receptor mediated endocytosis leading to endolysosomal sequestration. This was also investigated for PD-L1 targeted iTPALs. Following a 24 h incubation with iTPALs (17α), CT1BA5 cells were incubated further with LysoTracker™ Green DND-26 and imaged using confocal microscopy. The intrinsic fluorescence of BPD-PC in the iTPALs (17α) was used for imaging. FIG. 7 shows Confocal microscopy images of CT1BA5 cells using a 100× objective after 24 h incubation with iTPALs (17α) (left). Lysosomes are labeled with LysoTracker™ Green DND-26 (middle). The first two images (left and middle) were merged on ImageJ to identify regions of colocalization between iTPALs (17α) and lysosomes (right).

As shown in FIG. 7, iTPALs (17α) appeared as puncta inside the cell suggesting an endosomal internalization pathway, as opposed to membrane fusion which is also possible for liposomes. This was corroborated by the colocalization of iTPALs (17α) with the corresponding LysoTracker™ Green DND-26 as shown in FIG. 7 (right). This suggests that PD-L1 targeting of iTPALs results receptor mediated endocytosis and endolysosomal sequestration. Following internalization, it is very likely that PD-L1 and α-PD-L1 antibodies are degraded in the endolysosomes as is typical with all PD-L1 targeted immune checkpoint inhibitors. However it is also important to note here that reducing surface expression of PD-L1 using iTPALs is also of added benefit as the immune checkpoint becomes downregulated.

### Molecular Targeted Phototoxicity

By capitalizing on their PD-L1 specific internalization in cancer cells, we investigated the phototoxicity of iTPALs in a panel of murine cell lines, as summarized in Table 2 below. The cells were first incubated with a range of concentrations of PALs and iTPALs (17α) for 9 h and irradiated using 690 nm light with 25 mW/cm² irradiance and 40 J/cm² fluence. MTT metabolic activity assays conducted 48 h following irradiation were used to derive IC50 values for all treatments.

TABLE 2

| | IC₅₀ (nM) with iTPALs | IC₅₀ (nM) | % Reduction in IC₅₀ |
|---|---|---|---|
| Cell Lines | (17α) | with PALs | with targeting |
| MC38 | 424.9 | 560.6 | 24.2 |
| BMFA3 | 424.9 | 592.4 | 28.3 |
| LLC | 306.0 | 492.6 | 37.9 |
| AT84 | 1120.0 | 2208.0 | 49.3 |
| ID8 | 15.4 | 36.80 | 58.2 |
| CT1BA5 | 162.8 | 458.5 | 64.5 |

Phototoxicity was improved most in CT1BA5 cells 64.5% with PD-L1 targeting. Additionally, phototoxicity in MC38, BMFA3, LLC, AT84 and ID8 was improved by 24.2%, 28.3%, 37.9%, 49.3% and 58.2%, respectively with PD-L1 targeting. This result demonstrates that phototoxicity is not entirely dependent on PD-L1 expression levels or on targeted iTPAL (17α) binding. This is not unexpected, as sensitivities to PDT varies considerably between cell lines, and differences in sensitivities may be more dominant than the amount of iTPALs (17α) taken up by the cells.

### Inhibition of Pd-1/Pd-L1 Axis

As PD-L1 on the surface of cancer cells binds with PD-1 on the surface of T cells to suppress the T cell-mediated immune attack, α-PD-L1 antibodies have been clinically approved for their use in blocking the PD-1/PD-L1 axis. α-PD-L1 antibodies are also under investigation for PDAC, although responses have been limited. Although α-PD-L1 antibodies are FDA approved, immune related adverse events in 80% of patients with immune checkpoint monotherapies and in 95% of patients with combinational immune checkpoint therapy can oftentimes halt treatment. It is therefore important to develop strategies that lower the toxicity of α-PD-L1 immune checkpoint blocking without compromising their efficacy. Here, a single PD-L1 targeted iTPAL (17α) has been developed with the potential to target PD-L1 on cancer cells and simultaneously block the PD-L1/PD-1 axis.

To mimic PD-1 expressing T cells in vitro, recombinant mouse PD-1 was tethered to 6 m green fluorescing microspheres using streptavidin-biotin binding. The PD-1 microspheres were added to CT1BA5 cells in growth media, and to CT1BA5 cells that were pre-incubated with growth media and untargeted PALs, iTPALs (17α), or free α-PD-L1 antibodies at the same antibody equivalent concentration as the iTPALs (17α). The total number of PD-1 microspheres bound to the CT1BA5 cells were quantified over multiple ROIs. These numbers were then all normalized to the mean number of PD-1 microspheres bound to control CT1BA5 cells in growth media.

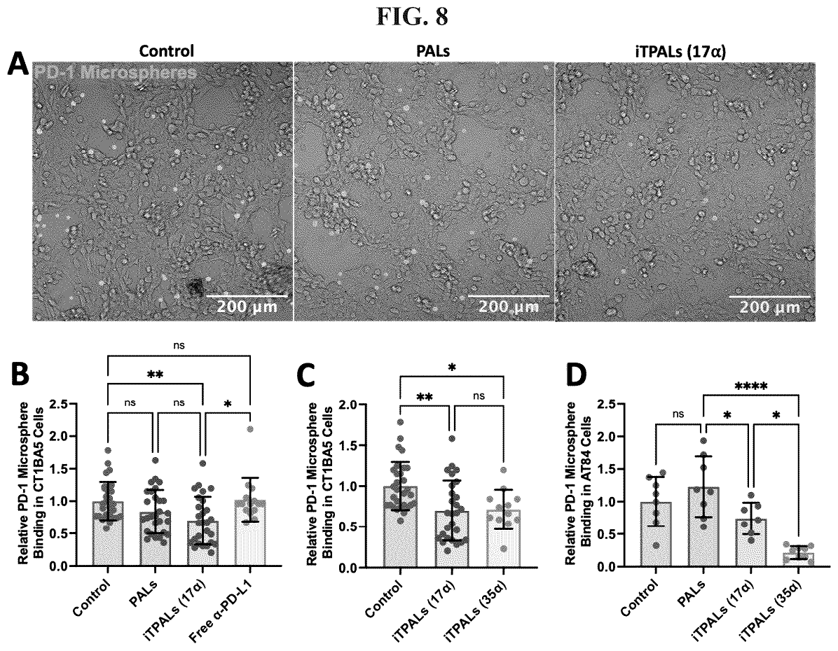

FIG. 8 shows that iTPALs block the PD-1/PD-L1 immune checkpoint. FIG. 8A shows representative confocal microscopy images showing cytotoxic T cell-mimicking microspheres tagged with recombinant mouse PD-1 binding to CT1BA5 cells (left). The cells were also pre-incubated with PALs (middle) and iTPALs (17α) (right) to assess the blocking of PD-1 microsphere binding to PD-L1 expressing CT1BA5 cells. FIG. 8B shows relative PD-1 microsphere binding to CT1BA5 cancer cells demonstrating that iTPALs (17α) are more effective at inhibiting PD-1/PD-L1 contact than free α-PD-L1 at the same antibody equivalent (7.5 nM). Increasing the ratio of α-PD-L1 per iTPALs from 17 to 35 does not further inhibit PD-1/PD-L1 contact in CT1BA5 cells (FIG. 8C) but does further inhibit PD-1/PD-L1 contact in AT84 cells (FIG. 8D). (Data are mean±S.D.; statistical significance was calculated using one-way ANOVA with a Tukey post-test on GraphPad Prism v 9.2.0, *: P<0.05, **: P<0.01, ****: P<0.0001).

As shown in FIGS. 8A and 8B, the PD-1 microsphere count was significantly reduced in CT1BA5 by 30.1% in the group pre-treated with iTPALs (17α). Untargeted PALs did not significantly reduce PD-1 microsphere binding, suggesting that non-specific blocking of the PD-1/PD-L1 axis was not significant. As such, the iTPALs (17α) effectively and specifically block the PD-1/PD-L1 axis. In contrast, free α-PD-L1 antibodies did not block the PD-1/PD-L1 axis when incubated with the cells at the same antibody equivalent as the iTPALs (17α). This difference suggests that multi-valent effects resulting from the multiple antibodies on each iTPALs (17α) can amplify the immune checkpoint blocking activity of the construct. Interestingly enough, the higher antibody density in the iTPALs (35α) construct did not further improve the blocking of the PD-1/PD-L1 axis in the CT1BA5 cells (FIG. 8C).

As a comparison, the ability of iTPALs to block of the PD-1/PD-L1 axis in AT84 cells was also investigated. iTPALs (17α) were able to block PD-1 microsphere binding 39.8% more than PALs (P<0.05) (FIG. 8D). Interestingly, iTPALs (35α) were 71.2% more effective than iTPALs (17α) at blocking PD-1 microsphere binding to AT84 cells. The reason for this discrepancy between CT1BA5 cells and AT84 cells is unclear and is likely to be a result of different PD-L1 receptor dimerization and clustering behaviors between the two cell lines, which is independent of PD-L1 expression levels.

Although not a perfect in vitro model for PD-1/PD-L1 axis, these findings suggest that iTPALs can be effective blockers of the immune checkpoint as well targeted delivery vehicles. By being able to block the PD-1/PD-L1 axis more efficiently than free α-PD-L1 antibodies, it is conceivable to predict that lower administered doses of α-PD-L1 antibodies, when tethered to iTPALs, can maintain their efficacy in vivo. These results underscore the potential for iTPAL (17α) constructs to minimize the risk of immune related adverse effects without compromising the efficacy of blocking the PD-1/PD-L1 axis.

### Immunogenic Cell Death (ICD)

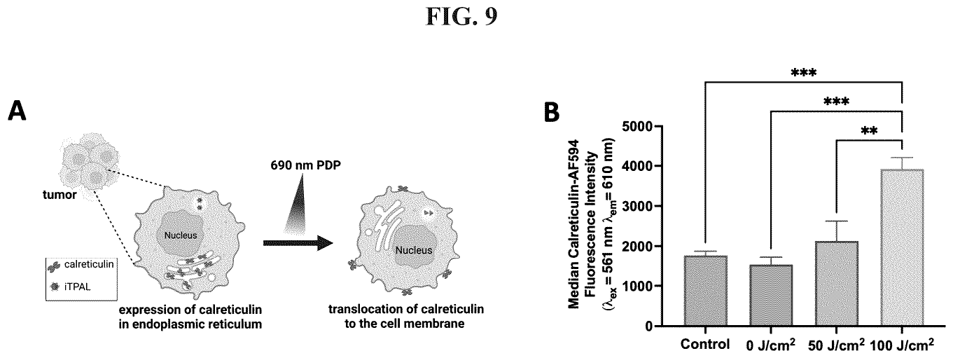

As described earlier, PDP of the tumor microenvironment can induce ICD which can improve the immunogenicity of the tumor, and ultimately its response to immunotherapy. FIG. 9 shows that iTPALs induce immunogenic cell death in PDAC cells. FIG. 9A shows a schematic representation of PDP-induced translocation of calreticulin (CRT) from the endoplasmic reticulum to the cell surface as a marker of immunogenic cell death (Created with BioRender.com). FIG. 9B shows an increase in the surface exposure of calreticulin in CT1BA5 cells is light dose dependent following incubation with iTPALs (17α). The median fluorescence intensity was obtained using flow cytometry after background subtraction of baseline fluorescence signals. (Data are mean±S.D.; statistical significance was calculated using one-way ANOVA with a Tukey post-test on GraphPad Prism v9.2.0, **: P<0.01, **P<0.001).

ICD is characterized by the release/translocation of different DAMPs including calreticulins (CRTs) as shown in FIG. 9A. The photoactivation of PSs induces immunogenic cell death through the release/translocation of different DAMPs. In this study, for the first time, the ability of the iTPALs (17α) to induce immunogenic cell death in CT1BA5 cells following 690 nm irradiation was demonstrated. For this, the translocation of CRTs to the cell surface lumen of the endoplasmic reticulum (ER) following 690 nm irradiation was explored in live cells and was found to be fluence-dependent (FIG. 9B). At 100 J/cm², 24 h post 690 nm irradiation, the translocation of CRTs was 2.2-fold higher than control, 2.6-fold higher than non-irradiated cells and 1.8-fold higher than 50 J/cm². This translocation of CRTs to the cell surface is known to be presented as a signal for phagocytic cells like dendritic cells and macrophages to engulf and present the tumor-associated antigens to T cells. Hence, this highlights the potential for iTPALs to also induce an adaptive immune response post PDP against PDAC tumors.

### Photomodulation of Collagen Hydrogels

The dense ECM in PDAC is a major hindrance for drug infiltration, especially for nanoparticles, and contributes to increased treatment resistance, tumor progression, and immunosuppression. Collagen is the most abundant ECM protein in PDAC and contributes to 90% of the ECM signaling in tumors. PDP-induced remediation of desmoplasia (reduction in collagen density and alignment) is correlated with improved survival. It is also known that the solid stress due to the excessive ECM proteins in desmoplastic tumors leads to the collapsing of blood vessels and limits the penetration of drugs into the tumors. In a study conducted in an in vitro Matrigel system, diffusion of antibody-targeted nanoparticles was significantly lower than untargeted nanoparticles. Herceptin targeted nanoparticles were not able to penetrate enough to access the cancer cells embedded in the Matrigel. As such, the dense ECM in PDAC clearly restricts the penetration of antibody-targeted nanoparticles, including the iTPALs, and can likely do so even more than for untargeted nanoparticles. As such, it is critical for nanoparticles to be able to modulate the stromal in PDAC to improve penetration. It is even more critical for a nanoconstruct to exhibit enhanced self-delivery through PDAC tumors to maintain a single-agent protocol and thus simplify its clinical translation.

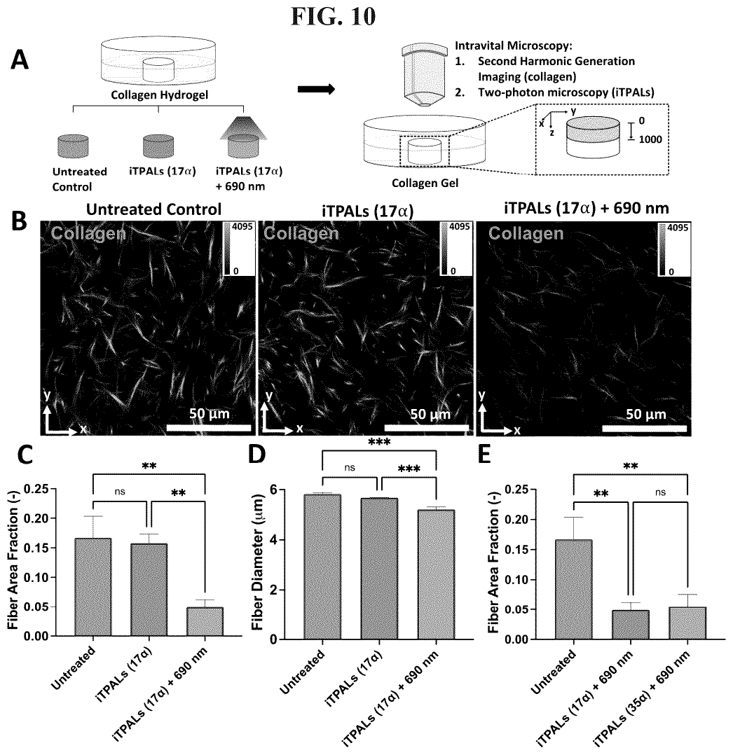

Considering that the presence of antibodies at the surface of nanoparticles significantly prevents their penetration through the ECM, the ability for iTPALs (17α) to self-penetrate through collagen hydrogels following light activation was explored. Polymerized 3D collagen hydrogels were incubated with iTPALs (17α) for 1 h, irradiated with 690 nm light, and imaged with second harmonic generation (SHG) imaging (collagen) and Two Photon Excitation imaging (iTPAL two photon fluorescence). FIG. 10 shows that PDP using iTPALs photomodulates collagen. FIG. 10A shows a schematic representation of the photomodulation of collagen hydrogels followed by imaging. FIG. 10B shows representative SHG images of collagen hydrogels on the untreated control (left), with iTPALs (17α) incubation and no irradiation (middle), and with iTPALs (17α) incubation and 690 nm irradiation (right). Graphs depict the reduction in collagen fiber area fraction (FIG. 10C), collagen fiber density (FIG. 10D), and comparison of reduction in collagen fiber area fraction with iTPALs (17α)+690 nm irradiation and iTPALs (35α)+690 nm irradiation (FIG. 10E) demonstrating the physical effects of iTPALs (35α)+690 nm on collagen. Images were quantified using the image segmentation tool CT-FIRE and analyzed on the topmost slice of the z-stack. (Data are mean±S.D.; statistical significance was calculated using one-way ANOVA with a Tukey post-test on GraphPad Prism v9.2.0, *: P<0.05, **: P<0.01, ****: P<0.0001).

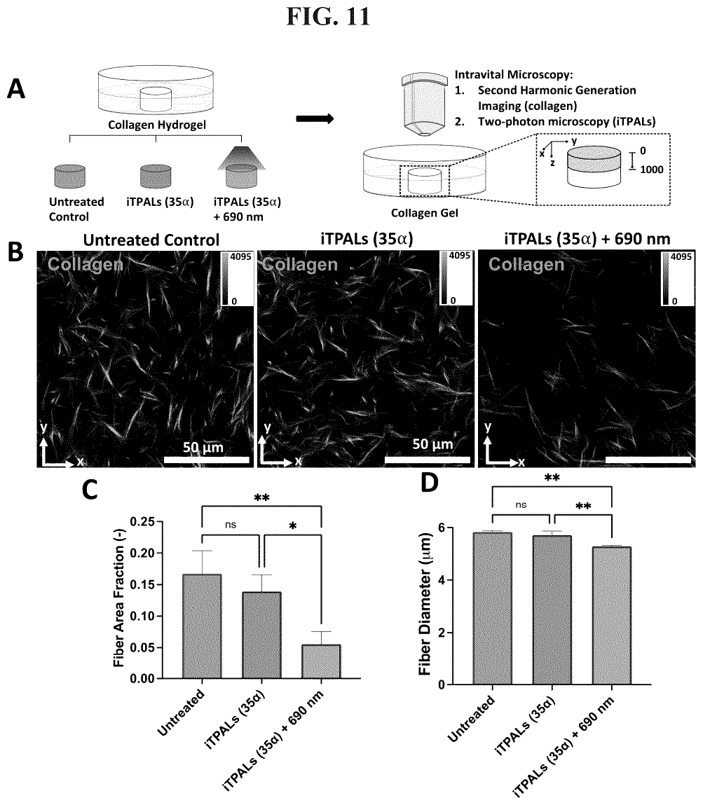

Following 690 nm light irradiation, the collagen matrices were found to be significantly disrupted as shown in FIG. 10B. This was further confirmed by the quantitative analyses on the first plane of collagen hydrogels, which revealed that photoactivation of iTPALs (17α) reduced the collagen fiber area fraction by 70.8% (FIG. 10C) and collagen fiber diameter by 10.6% (FIG. 10D). The collagen photomodulation with iTPALs (35α) that have a higher antibody density at their surface was also explored. However, the reduction in fiber area fraction with iTPALs (35α) was comparable to that of iTPALs (17α) (FIG. 10E, FIG. 11A-11D). The PS BPD used in this study is known to produce singlet oxygen, hydroxyl radicals and superoxide anions when excited with 690 nm light. It is known that the singlet oxygen selectively oxidizes collagen crosslink histidinohydroxylysinonorleucine and its precursor histidine, whereas hydroxyl radicals and superoxide anion attack proline or 4-hydroxyproline residues in collagen. Although studies have already shown how photochemical species, such as those produced by iTPAL activation, the ability for photoactivated PS-containing agents, such as iTPALs, to exhibit improved self-delivery through dense ECM following irradiation has not been demonstrated before.

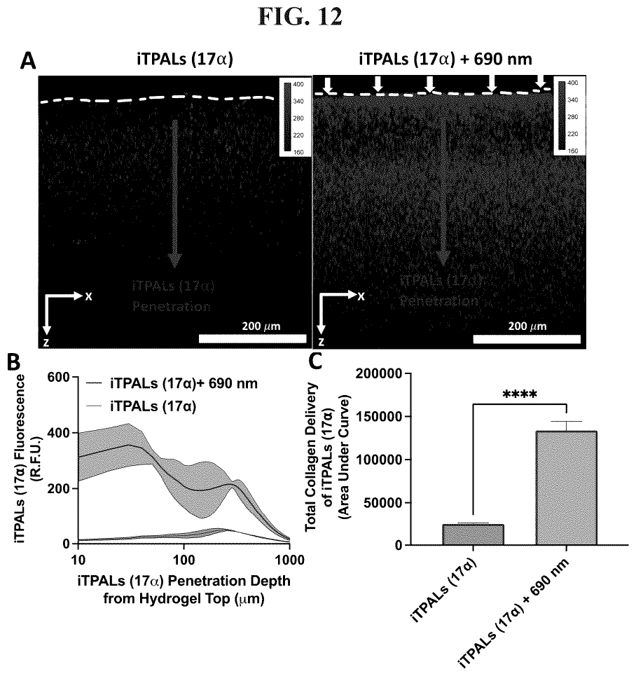

Here, it is shown for the first time that the priming of collagen hydrogel promotes the self-penetration of iTPALs (17α), thus showing potential in improving their own delivery through desmoplastic tumors. This was evident from the two-photon microscopy images which showed significantly higher signals from iTPALs (17α) in the hydrogel following irradiation, as compared to non-irradiated hydrogels. FIG. 12 shows that iTPALs promote self-delivery through collagen following photoactivation. FIG. 12A shows a representation of the self-delivery of iTPALs (17α) through collagen that is potentiated by 690 nm irradiation. Fluorescence of iTPALs (17α) obtained by two-photon excitation imaging. FIG. 12B shows quantification of the penetration of iTPALs (17α) through the collagen hydrogel from the top where they were added to the hydrogel. FIG. 12C shows a graph demonstrating the greatest improvement in accumulation of iTPALs (17α) into the collagen hydrogel following 690 nm irradiation. Image quantification was performed by averaging the signal intensity on the plane of interest using a custom MATLAB script. (Data are mean±S.D.; statistical significance was calculated using unpaired t-test on GraphPad Prism v9.2.0, n=3, ****: P<0.001).

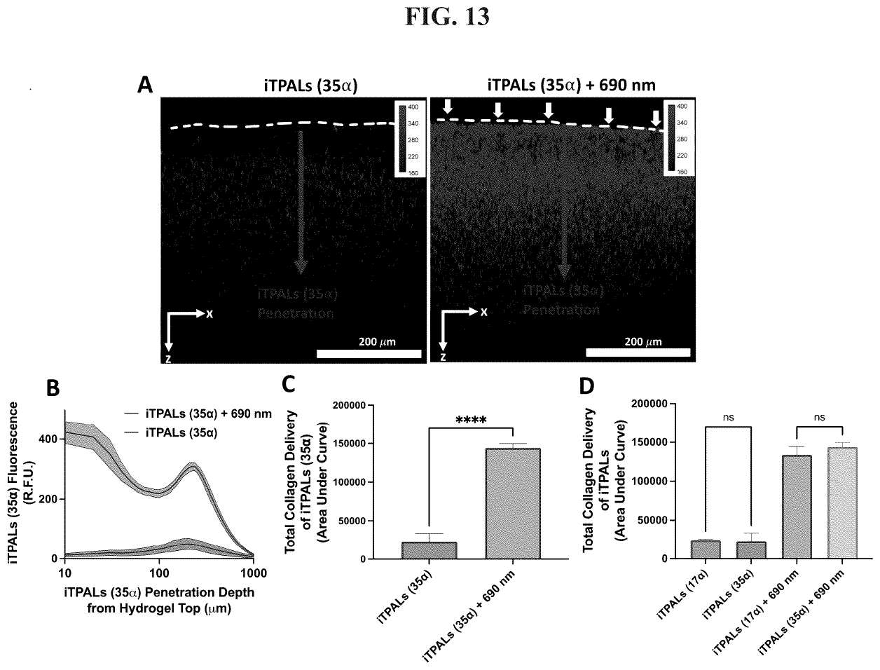

The self-penetration of iTPALs (17α) was quantified and it was found that the total accumulation of iTPALs (17α) was increased by 5.4-fold (FIG. 12B) post 690 nm light activation. This is attributed to the oxidative photomodulation of collagen which improves its permeability to the iTPALs. The self-penetration of iTPALs (35α) that have a higher antibody density as a control was explored and a comparable improvement in self-delivery through collagen was seen (FIG. 13A-13D). This suggest that self-delivery by PDP may be agnostic to the number of antibodies attached to iTPALs, and potentially other nanoconstructs. While these studies provide insights into the contribution of collagen photomodulation to self-delivery of iTPALs, they do not account for the impact of other tumor ECM molecules, the cellular composition of the tumor, or the vascular fluid dynamics and mechanical stress in the tumor. The presence of iTPALs alone without 690 nm irradiation had no impact on the collagen. Overall, these results highlight the ability of iTPALs to effectively disrupt collagen networks and facilitate their own self-penetration. This phenomenon could prove critical in improving the co-delivery of PDP agents, chemotherapeutics and immune checkpoint inhibitors.

### Longitudinal In Vivo Tumor Imaging

PDP has been known over a decade to prime the tumor microenvironment and improve drug delivery to the tumor. There is a dramatically improved delivery of liposomal formulation of doxorubicin drug (Doxil) to subcutaneous tumors with PDP. The interstitial tumor vessels in PDP treated A431 xenografts in mice were shown to be dilated and permeabilized. This resulted in a greater accumulation of liposomal daunorubicin (DaunoXome) as well as antibody-drug conjugate (ADC) within tumors. PDP also improves the delivery and intratumoral distribution of tumor targeting antibodies in head and neck tumors. Similarly, PDP has been demonstrated to increase the local concentration of Onivyde in PDAC tumors which prolonged the survival in mice. However, all these studies have been attempted by administering a photodynamic agent and a therapeutic agent separately which adds complications to their clinical translation.

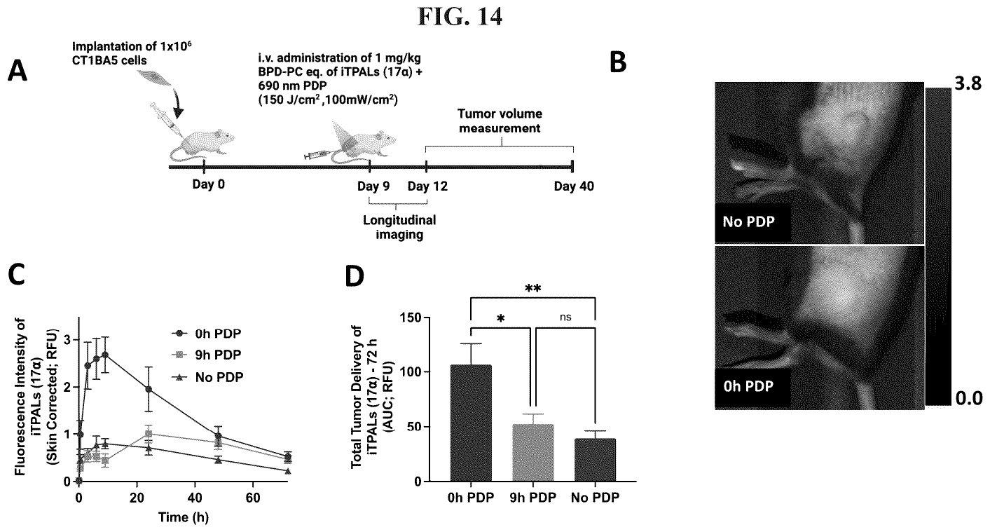

This study is the first of its kind to have a rapidly translatable single-agent protocol to prime the tumor microenvironment using PDP and improve the delivery into the PDAC tumors. This approach is geared towards augmenting the treatment response of chemo-immunotherapy combinations in future. As such, the capacity for PDP-induced self-delivery of iTPALs (17α) was examined in vivo in subcutaneous CT1BA5 tumors immediately after administration (0 h PDP) and PDP 9 h after administration using 690 nm light with a fluence of 150 J/cm² and an irradiance of 100 mW/cm². FIG. 14 shows that iTPALs promote self-delivery through PDAC tumors following photoactivation. FIG. 14A shows a schematic representation of the timeline of the animal study conducted in C57BL/6 mice bearing CT1BA5 tumors. FIG. 14B shows representative images of the mice bearing CT1BA5 tumors, administered with iTPALs (17α) without (top) and with (bottom) PDP using 690 nm irradiation at 0.5 h following intravenous administration. FIG. 14C shows quantification of longitudinal tumor fluorescence imaging depicting the delivery and retention of iTPALs (17α) with 0 h, 9 h PDP and without PDP. FIG. 14D shows Area Under Curve (AUC) analyses showing the total accumulation of iTPALs (17α) into the tumor as calculated using GraphPad Prism v9.2.0. (Data are mean±S.E.M.; statistical significance was calculated using one-way ANOVA with a Tukey post-test on GraphPad Prism v9.2.0, *: P<0.05, **: P<0.001).

As shown in FIGS. 14B and 14C, PDP at 0 h using intravenously administered iTPALs (17α) results in a significant improvement in self-delivery in the CT1BA5 tumors, while PDP at 9h after administration has little effect on self-delivery of iTPALs. 0 h PDP improved the self-delivery of iTPALs (17α) by up to 237.6% after administration and irradiation (FIG. 14C). Similarly, 0 h PDP improved the total bulk tumor accumulation of iTPALs (17α) over 72 h by 172.9% as shown in the AUC analyses (FIG. 14D). However, 9 h PDP did not significantly improve the tumor self-delivery of iTPALs (17α). These findings suggest that 0 h PDP is superior to PDP with a 9 h interval at priming the tumor microenvironment. 0 h PDP ensures that sufficient iTPALs (17α) are in circulation that can actually benefit from the priming and self-delivery. This is most likely to be attributed to the photomodulation of tumor collagen in addition to improved vascular permeability to iTPALs (17α), as has been reported using various theranostic agents. This desmoplasia-remediating construct shows considerable promise for PDP-based combination therapies that leverage its immune checkpoint targeting and blocking phenomena, as well as enhanced self-delivery through desmoplastic tumors.

### In Vivo Photodynamic Priming of Aggressive PDAC Tumors

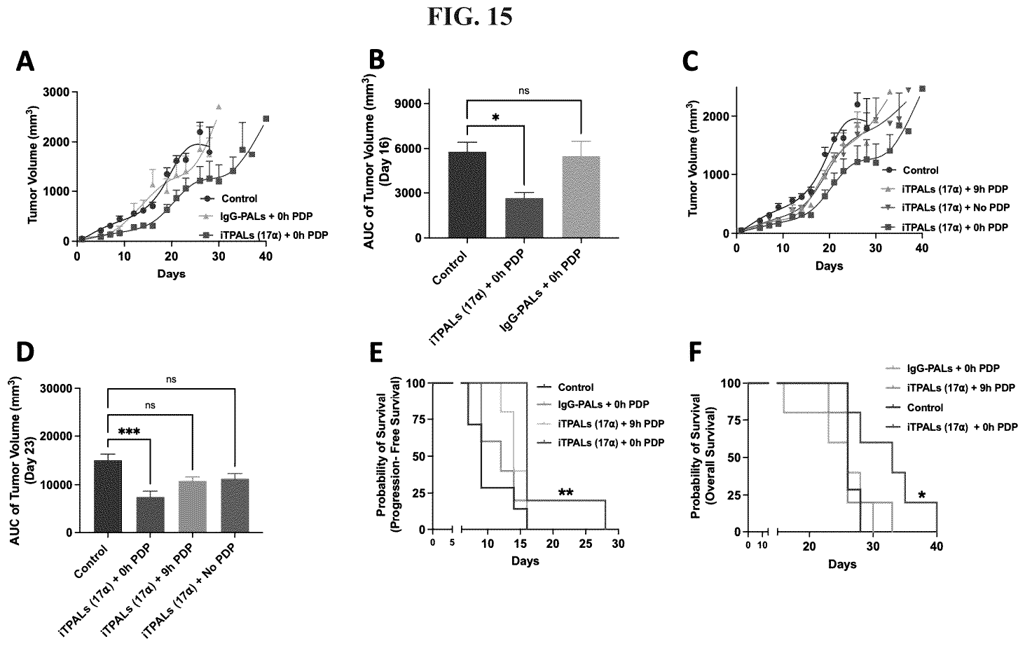

In clinical studies, immunotherapy is typically given over multiple cycles when combined with secondary treatment modalities, such as radiotherapy and chemo-radiotherapy (NCT02318771, NCT02492568, NCT02764593, NCT02525757). FIG. 15 shows that a single PDP dose using iTPALs (17α) controls CT1BA5 PDAC tumor growth and prolongs the survival. Table 3 below shows the dosimetry used for the in vivo PDP study.

TABLE 3

| | BPD-PC | Drug-Light | Light Fluence at |
|---|---|---|---|
| | eq. dose | Interval | 100 mW/cm² |
| Treatment Arms | (mg/kg) | (DLI; h) | (J/cm²) |
| Control | 0 | — | 0 |
| iTPALs (17α) | 1 | 0 | 150 |
| iTPALs (17α) | 1 | 9 | 150 |
| IgG-PALs (sham) | 1 | 0 | 150 |
| IgG-PALs (sham) | 1 | 9 | 150 |

FIG. 15A shows a spline plot of the tumor volumes of mice until day 40 when all mice died, FIG. 15B shows area under curve of tumor volumes until day 16 (when a first mouse from the groups died) depicting a delay in tumor growth when treated with a single dose PDP (0 h) using iTPALs (17α) in comparison to sham IgG-PALs (0 h PDP) and the untreated control. FIG. 15C shows a spline plot of the tumor volumes of mice until day 40 when all mice died, and FIG. 15D shows area under curve of tumor volumes until day 23 (when the first mouse from the respective treatment groups died) depicting a delay in tumor growth when treated with a single dose of PDP (0 h) using iTPALs (17α) in comparison to iTPALs (17α) with 9 h PDP, iTPALs (17α) without PDP, and the untreated control. FIG. 15E shows Kaplan-Meier plots representing the probability of progression free survival (tumor volume <500 m³) in mice where iTPALs (17α) with 0 h PDP significantly improves the progression free survival in mice. FIG. 15F shows Kaplan-Meier plots representing the probability of overall survival in mice where iTPALs (17α) with 0 h PDP significantly improves the overall survival in mice. (Data are mean±S.E.M.; statistical significance was calculated using one-way ANOVA with a Tukey post-test on GraphPad Prism v9.2.0; statistical significance for survival data was calculated using Log-rank (Mantel-cox test) on GraphPad Prism v9.2.0; *: P<0.05, **: P<0.01, ***: P<0.001).

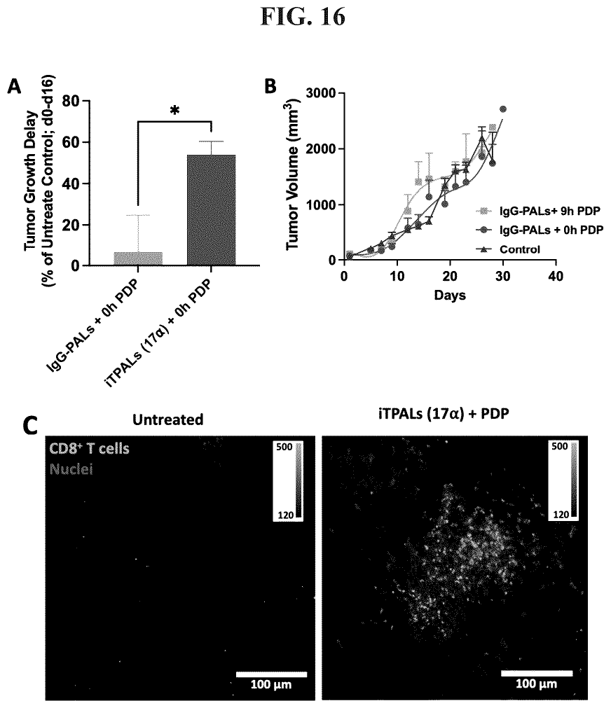

FIG. 16A shows that umor growth is significantly delayed when tumors were treated with 0 h PDP using the iTPALs (17α) in comparison to 0 h PDP using sham IgG-PALs. Tumor growth delay is presented as a percentage of cumulative tumor growth between day 0 and day 16 (prior to any animal deaths in any cohort), with respect to cumulative tumor growth of untreated control animals. FIG. 16B shows tumor growth curves in mice treated with PDP using IgG-PALs at different drug light interval (0 h, 9 h) until day 30 when all mice in the groups died. FIG. 16C shows immunofluorescence staining of CT1BA5 tumors 7 days after 0 h PDP using the iTPALs (17α) reveals an increase in tumor infiltration of CD8⁺.

In this study, with even just a single sub-curative priming dose of iTPAL (17α) 0 h PDP, the growth of aggressive CT1BA5 tumors was inhibited by 54.1% (FIG. 15A, 15B, FIG. 16). Conversely, using equivalent sham IgG-PALs that have no affinity for PD-L1, which are also activated with the same dose parameters of 690 nm light immediately following administration, do not significantly inhibit tumor growth. It should also be noted that PD-L1 targeting using iTPALs (17α) without light activation did not provide any significant delay in tumor growth, thus underscoring the cooperative effects of PDP and PD-L1 blockade. Similarly, as shown in FIG. 15B, while PDP at 0 h using iTPALs (17α) significantly inhibited tumor growth, PDP at 9 h using iTPALs (17α) did not delay tumor growth. PDP at 0 h using iTPALs (17α) provided a greater degree of self-delivery than PDP at 9 h. A statistically significant direct correlation was observed between tumor self-delivery following PDP (0 h and 9 h) and overall survival in mice treated with iTPALs (Pearson's r=0.670 P=0.034). This improvement in efficacy at 0 h is, therefore, directly related to the significantly improved self-delivery of iTPALs (17α) with 0 h PDP in comparison to 9 h PDP, as demonstrated in FIGS. 15C and 15D. No correlation between tumor delivery of sham IgG-PALs and overall survival was found (P=0.155), confirming that the improved overall survival was a direct result of immune checkpoint blockade using iTPALs that was augmented by PDP.

Median survival, progression-free survival and overall survival in mice with iTPALs (17α) were also evaluated. PDP at 0 h improved the median survival in mice by 26.9%, progression-free survival by 75.0% and overall survival by 42.9% (FIG. 15E, 15F). Results are shown in Table 4 below.

TABLE 4

| | Median | Progression- | Overall |
|---|---|---|---|
| | Survival | Free Survival | Survival |
| Treatment Arm | (days) | (days) | (days) |
| Control | 26 | 16 | 28 |
| iTPALs (17α) + 0 h PDP | 33 | 28 | 40 |
| iTPALs (17α) + 9 h PDP | 26 | 16 | 33 |
| IgG-PALs + 0 h PDP | 26 | 16 | 30 |
| IgG-PALs + 9 h PDP | 23 | 12 | 28 |

However, with IgG-PALs, the median survival, progression free survival and overall survival did not improve, which highlights the importance of targeting and blocking PD-L1 using iTPALs (17α).

Similarly, for 9 h PDP and no-light iTPALs (17α) controls, no significant improvements were found for median survival, progression-free survival, and overall survival in mice. This is attributed to the significant improvement in the self-delivery of iTPALs (17α) when activated immediately after administration, as compared to 9 h after administration. Only a single sub-curative PDP dose significantly extends survival in mice bearing PDAC tumors. When applied over multiple cycles, as is typical for immune checkpoint therapy, PDP using iTPALs (17α) that contain chemotherapy promises to offer durable tumor growth inhibition and significant survival benefit in PDAC.

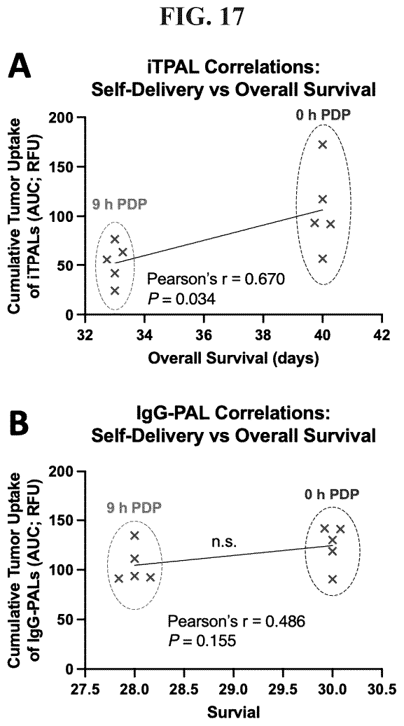

FIG. 17 shows that iTPAL self-delivery directly correlates with improved overall survival. Scatter plots of the cumulative tumor uptake of iTPALs (FIG. 17A) and sham IgG-PALs (FIG. 17B) following PDP reveal a statistically significant correlation between iTPALs self-delivery and overall survival (P=0.034). No relationship between tumor delivery of IgG-PALs and overall survival is found. (P values (two-tailed test) correspond to the statistical significance of the Pearson's r correlation calculated using GraphPad Prism v9.2.0.).

### Analysis of the Tumor Microenvironment

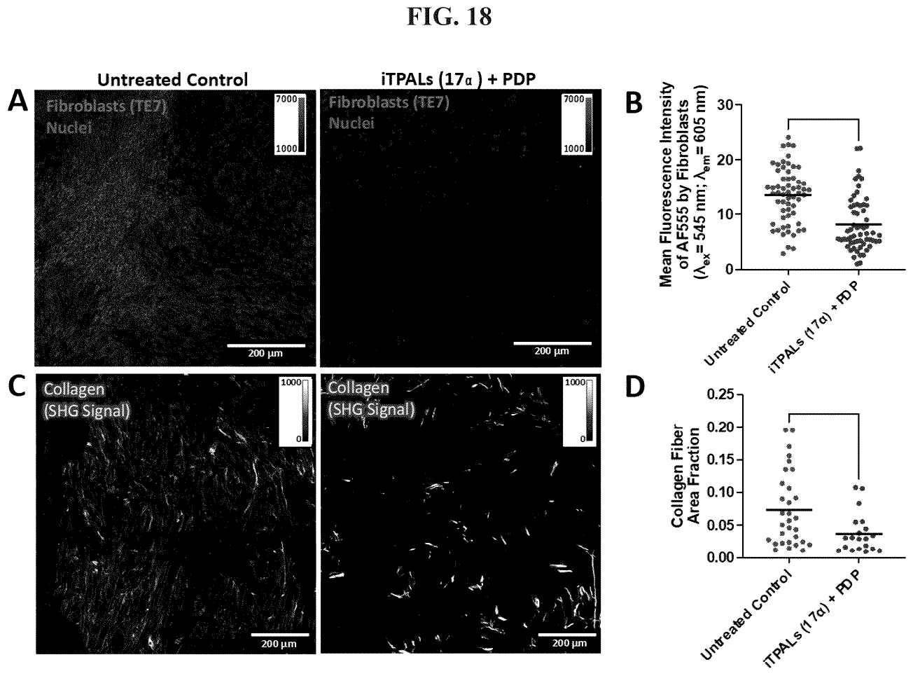

It has been shown that fibroblasts were more sensitive to being depleted following PDT in heterocellular 3D nodules containing PDAC cells. Similarly, PDT of adventitial fibroblasts decreases their migration and proliferation. As such, effects of PDP using iTPALs (17α) on fibroblasts in PDAC tumors at 72 h following treatment were explored. FIG. 18 shows that a single PDP dose using iTPALs (17α) reduces fibroblast content and collagen density in CT1BA5 PDAC tumors. FIG. 18A shows representative immunofluorescence images depicting decreased fibroblast contents on the cyrosectioned CT1BA5 tumor tissues when treated with iTPALs (17α)+PDP. FIG. 18B shows quantitative analysis of fluorescence specific to fibroblasts from 10 ROIs spanning the entire tumor cross-section from 6 tumor samples for each condition. Mean fluorescence is calculated using Image J. FIG. 18C shows representative second harmonic generation (SHG) images of cryosectioned CT1BA5 tumor tissues showing reduced collagen area fraction when treated with iTPALs (17α)+PDP. FIG. 18D shows quantitative collagen area fraction on the control tumor tissues and iTPALs (17α)+PDP treated tumor tissues depicting significant reduction in collagen fiber area fraction with the treatment. (Data are mean±S.E.M.; statistical significance was calculated using a two-tailed t test on GraphPad Prism v9.2.0; *: P<0.05, ****: P<0.0001).

Immunofluorescence staining revealed that PDP using iTPALs (17α) reduces the tumor fibroblast content by 39.4% (FIG. 18A, 18B). Tumor collagen was also quantified at 72 h following treatment using SHG imaging. The study revealed that PDP using iTPALs (17α) reduces the collagen area fraction significantly by 49.1% (FIG. 18C, 18D). Remediating desmoplasia by modulating the tumor stroma promises to improve PDAC responses to chemotherapy, radiotherapy, and immunotherapy. It has been shown that PDP using a liposomal formulation of BPD (the same PS in iTPALs) induces immunogenic cell death in tumor tissue in vivo. Here, PDP using iTPALs appears to increase CD8⁺ T cell infiltration in PDAC tumors.

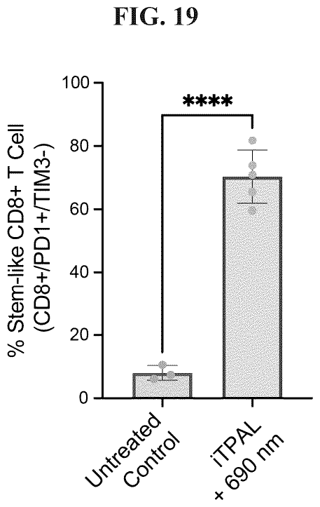

Whole Tumor Flow Cytometry. CT1BA5 cells (1×10⁶ cells) in 20 μl of sterile DPBS were implanted subcutaneously into the left flank of male C57BL/6 mice (20 g, 6 weeks old). When the tumors reached a volume of 300-500 mm³, the mice were divided into two cohorts: control and iTPALs (17α)+690 nm group. The mice were then intravenously administered with 2 mg/kg of iTPALs (BPD-PC equivalent). Immediately following administration, tumors in the iTPALs (17α)+690 nm group were irradiated with a 690 nm laser at a fluence of 50 J/cm² and an irradiance of 50 mW/cm². Eight days following administration, tumors were extracted and homogenized to isolate leukocytes. The leukocytes were then labeled with an antibody cocktail containing anti-CD8 (Clone: 53-6.7), anti-PD-1 (Clone: RMP 1-30), and anti-TIM3 (Clone: RMT3-23) antibodies. The antibodies were stained with fluorescently labeled secondary antibodies and flow cytometry was then performed to identify the percentage of stem-like CD8⁺ T cells (CD8⁺ PD-1⁺ TIM3⁻) in the tumors. FIG. 19 shows PDAC tumor infiltration of stem-like CD8+ T cells. Light activation of iTPALs causes a significant ˜9-fold increase in CT1BA5 pancreatic tumor infiltration of stem-like CD8+ T cells. Stem-like CD8+ T cells are a subset of T cells (CD3+) with superior persistence, anti-tumor immunity, and responsiveness to PD-L1 or PD-1 immune checkpoint blockade.

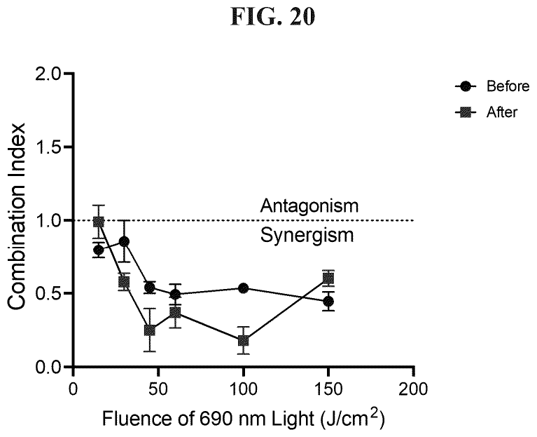

Combination Index. To calculate the synergy of iTPALs (17α) with nanoliposomal irinotecan in killing CT1BA5 cells, Combination Index analysis was used. The dose response of photodynamic therapy using iTPALs and nanoliposomal irinotecan on CT1BA5 cells was first measured using an MTT assay. To calculate the combination index (CI), CT1BA5 cells were treated with photodynamic therapy using iTPALs (17α) or with a combination of iTPAL (17α) photodynamic therapy and nanoliposomal irinotecan at an IC50 dose of 0.02 mg/ml. After 9 h of incubation, the cells were irradiated using 690 nm light at varying fluences (0.5 J/cm², 15.0 J/cm², 30 J/cm², 45 J/cm², 60 J/cm², 100 J/cm², 150 J/cm²). Nanoliposomal irinotecan added before or after photodynamic therapy using iTPALs (17α) was compared. The cells were then incubated for 72 h and analyzed using the MTT assay. The concentrations of drug added and the resultant percentage viabilities were then entered into Compusyn software to calculate the combination index. A combination index (CI)<1 suggests synergism, CI>1 suggests antagonism, and CI=1 suggests an additive effect. FIG. 20 shows that light activation of iTPALs in CT1BA5 cells synergistically reduces viability when combined with nanoliposomal irinotecan.

### Solid Lipid Nanoparticles

Solid lipid nanoparticles (LNP) were also explored for use in place of liposomes.

Synthesis of LNP BPD-PC and Lipo BPD-PC: Firstly, benzoporphyrin derivative (BPD) was conjugated to 1-arachidoyl-2-hydroxy-sn-glycero-3-phosphocholine (20:0 lyso PC) to yield lipid-anchored benzoporphyrin derivate (BPD-PC), according to our previously described methods. Briefly, BPD (US Pharmacopeia), 4-(Dimethylamino) pyridine (DMAP, Sigma-Aldrich), 1-ethyl-3-(3-dimethylaminopropyl) carbodiimide (EDC; Sigma-Aldrich), and N,N-Diisopropylethylamine (DIPEA, Sigma-Aldrich), 1-arachidoyl-2-hydroxy-sn-glycero-3-phosphocholine (20:0 Lyso PC, Avanti) were mixed at molar ratios of 1:5:50:25:60, respectively, in 5 mL of dichloromethane (DCM; Fischer Scientific, high-performance liquid chromatography [HPLC] grade) and stirred at 2500 RPM for 72 h at room temperature. BPD-PC was purified using a preparatory thin-layer chromatography and extracted in a 2:1 dichloromethane/methanol mixture. The extracted BPD-PC was finally filtered through a 0.22 μm polytetrafluoroethylene (PTFE) filter and stored in chloroform at −20° C. in the dark.

For LNP BPD-PC, all lipid components, including 1,2-Dipalmitoyl-sn-glycero-3-phosphocholine (DPPC), 1,2-Dimyristoyl-sn-glycero-3-phosphocholine (DMG-PEG), cholesterol, the ionizable lipid SM-102 9-heptadecanyl 8-{(2-hydroxyethyl)[6-oxo-(undecyloxy)hexyl]amino}octonate, and the 20:0 BPD-PC were mixed at a molar ratio of 0.090:0.015:0.385:0.500:0.01, respectively into a 5 mL glass vial(20,21). The lipids were dried using nitrogen gas until a lipid film was formed. After drying, 0.25 mL of ethanol was added to the lipid film and vortexed for 10 minutes at 2000 rpm. The lipid-ethanol mixture was added dropwise by a syringe pump pumping at a flow rate of 0.8 mL/min into the 0.75 mL of buffer solution. The buffer solution was prepared separately by dissolving 11.76 mg of sodium citrate tribasic (Sigma-Aldrich) dihydrate in 4 mL of MilliQ water, and the pH was adjusted to 4 using HCl (1 M, approximately 65 μL). Following the dropwise addition, the LNP solution was placed on a magnetic stirrer and stirred for 18 hours at 2500 rpm. After the 18-hour stirring period, the LNPs were subjected to dialysis by replacing the buffer solution with 1× DPBS using a 100 kDa dialysis tube. The dialysis tube was placed in a beaker containing 1 L of 1× DPBS prepared by diluting 100 mL of 10× DPBS with 900 mL of MilliQ water and stirred at 150 rpm. The DPBS solution was replaced three times within 24 hours, with intervals of at least 8 hours between each replacement. After the 24-hour dialysis process. The resultant LNP BPD-PC were stored at 4° C. in a dark environment for future use.

For the synthesis of Lipo BPD-PC, the lipids, 2-Dipalmitoyl-sn-glycero-3-phosphocholine (DPPC: Avanti), cholesterol,1,2-Distearoyl-sn-Glycero-3-Phosphoethanolamine with conjugated methoxyl poly (ethylene glycol) (DSPE-mPEG₂₀₀₀; Avanti), and 20:0 lyso-PC-BPD in chloroform were mixed at a molar ratio of 0.675:0.300:0.0015:0.010, respectively. The liposomes were prepared using a conventional thin-film hydration method by hydrating in Dulbecco's Phosphate Buffered Saline (DPBS; no Calcium, no Magnesium; Corning) followed by ultrasonication (Ultrasonic probe sonicator; Fisher Scientific) for a total of 30 mins (20 s on/40 s off cycles) at 42° C. in the dark.

Table 5 below shows a summary of the hydrodynamic diameter, polydispersity index, zeta potential of the developed nanoformulations. Data is mean±SD from three individual preparations for each nanoformulation.

TABLE 5

| | Hydrodynamic | Polydispersity | Zeta Potential |
|---|---|---|---|
| Nano-formulation | Diameter (nm) | index (P.D.I) | (mV) |
| LNP BPD-PC | 157.8 | 0.09 | −4.30 |
| Lipo BPD-PC | 107.1 | 0.13 | −1.56 |

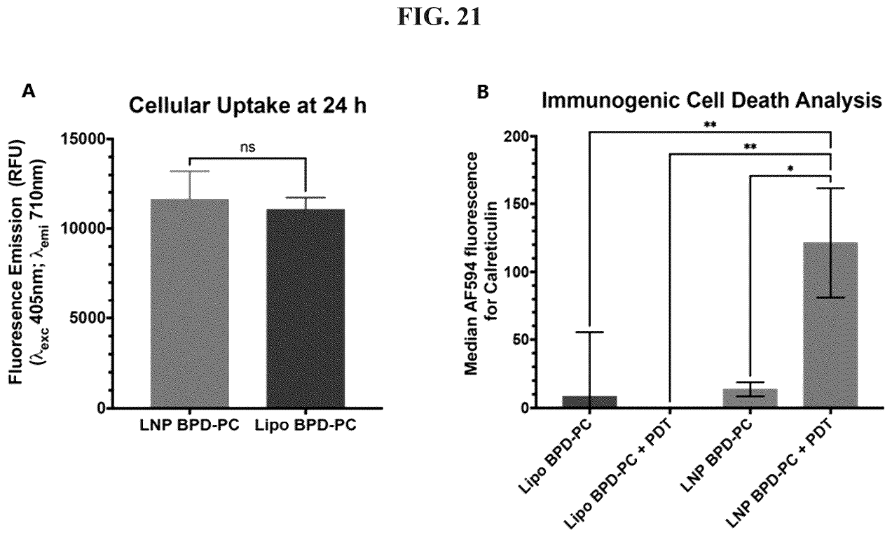

FIG. 21A shows cellular uptake of LNP BPD-PC and Lipo BPD-PC at 24 h in CT1BA5 cells. FIG. 21B shows expression of immunogenic cell death marker calreticulin in CT1BA5 cells following no PDT and PDT with LNP BPD-PC and Lipo BPD-PC with 20 J/cm² fluence. (Data are mean±S.D.; statistical significance was calculated on GraphPad Prism v9.2.0, *: P<0.1, **: P<0.01, ***: P<0.001).

Cellular uptake: For cellular uptake, 50,000 CT1BA5 cells were plated in a clear bottom transparent 96 well plate (Corning) and incubated at 37° C. for 24 h. After 24 h cells were incubated with 250 nM BPD-PC equivalent LNP BPD-PC and Lipo BPD-PC. After 24 h incubation cells were trypsinized and pelleted using centrifugation (250 g for 5 min). Further cells were resuspended in 300 μL of Fluorescence-Activated Cell Sorting (FACS) buffer (1% FBS in 1× DPBS) and transferred into flow cytometry tubes. Flow cytometry (BD Fortessa) analysis was done using a 405 nm laser and a 710/50 nm bandpass detector.

Cellular uptake of PS delivery vehicle is very crucial for PDT based treatments. The cellular uptake of LNP BPD-PC and Lipo BPD-PC was explored in CT1BA5 cells. As shown in FIG. 21A, results revealed that no significant difference existed between the uptake of LNP BPD-PC and Lipo BPD-PC, suggesting that any difference in size of LNP BPD-PC and Lipo BPC-PC had no impact on internalization. This result shows that both the nano formulations can work as equivalent intracellular drug delivery vehicles.

Immunogenic Cell Death: 45,000 CT1BA5 cells were seeded in a 6 well plate and incubated at 37° C. for 24 h. The media in each well was replaced with media containing 0.018 nM (IC₂₅) BPD-PC equivalent of Lipo BPD-PC and 0.011(IC₂₅) nM BPD-PC equivalent of LNP BPD-PC. After 24 h incubation, cells were irradiated with 690 nm light at an irradiance of 17.86 mW/cm² and fluence of 20 J/cm². Following 24 h cells were trypsinized and collected in eppendorf tubes and were washed thrice with DPBS. Cells in each eppendorf tube were fixed with 4% formaldehyde for 15 min at room temperature. Furthermore, cells were washed and resuspended in 100 μL of anti-calreticulin antibody (D3E6; Cell Signaling Technology). Cells were incubated for 1 h and washed again with DPBS. Then the cells were resuspended in 100 μL of secondary antibody conjugated with AF594 (Cell Signaling Technology). After 20 minutes of incubation in dark environment, cells were washed and resuspended in FACS buffer. Flow cytometer (BD Fortessa) analysis was done to calculate the median calreticulin fluorescence using a 561 nm laser and a 610/20 nm bandpass detector.

As discussed earlier PDT can induce immunogenic cell death (ICD) though release of damage-associated molecular patterns (DAMP's) such as calreticulin, HSP 60&70 and high mobility group box 1(HMGB1). A previous study also reported the ability of triple receptor targeted liposome formulation to induce ICD with 690 nm light irradiation up to 100 mW/cm²(19). The role of LNP BPD-PC and Lipo BPD-PC in inducing ICD was investigated by looking at calreticulin expression post irradiation at 17.86 mW/cm² with fluence of 20 J/cm² in CT1BA5 cells. As shown in FIG. 21B, LNP BPD-PC showed the highest expression of calreticulin post PDT. This suggests that LNP BPD-PC can induce an immune response post PDT in pancreatic cancer using lower PDT doses (20 J/cm²) than those needed to induce an immune response with liposomes (100 J/cm², FIG. 9B).

What is claimed is:

1. Photoactivatable nanoparticles capable of self-delivery for use as therapeutic agents in immunotherapy treatments, comprising:
nanoparticles;
at least one photosensitizer attached to the nanoparticles; and
at least one immune checkpoint targeting feature attached to the nanoparticles, wherein the immune checkpoint targeting feature inhibits an immune checkpoint in a tumor or cancer cell, and wherein the photoactivatable nanoparticles are self-delivered to the tumor or cancer cell through light activation of the photosensitizer.

2. The photoactivatable nanoparticles of claim 1, wherein the nanoparticles are liposomes, solid lipid nanoparticles, organic or inorganic nanoparticles, self-assembled nanoparticles, crystalline nanoparticles, amorphous nanoparticles, lipid micelles, polymer micelles, hybrid micelles, or combinations thereof.

3. The photoactivatable nanoparticles of claim 1, wherein the photosensitizer is BPD-PC.

4. The photoactivatable nanoparticles of claim 1, wherein the immune checkpoint targeting feature is an α-PD-L1 antibody.

5. The photoactivatable nanoparticles of claim 1, wherein up to 35 immune checkpoint targeting features are attached to the nanoparticle.

6. The photoactivatable nanoparticles of claim 1, wherein the nanoparticle is a liposome, wherein the photosensitizer is BPD-PC, wherein the immune checkpoint targeting feature is an α-PD-L1 antibody, and wherein seventeen α-PD-L1 antibodies are attached to the nanoparticle.

7. A pharmaceutical composition for use in inhibiting tumor growth in a patient comprising a therapeutically effective amount of the photoactivatable nanoparticles of claim 1 and a pharmaceutically acceptable excipient, adjuvant, carrier, buffer, stabilizer, or mixture thereof.

8. The pharmaceutical composition of claim 7, further comprising therapeutically effective amounts of one or more additional treatment agents, wherein the one or more additional treatment agents are chemotherapy agents, small molecular weight inhibitors, targeted drugs, nucleic acids, mRNA, DNA, radiosensitizers, or combinations thereof.

9. A method for inhibiting tumor growth in a patient having a tumor, comprising:
administering a pharmaceutical composition to the patient, wherein the pharmaceutical composition comprises photoactivatable nanoparticles, wherein the photoactivatable nanoparticles comprise nanoparticles, at least one photosensitizer attached to the nanoparticles, and at least one immune checkpoint targeting feature attached to the nanoparticles, and wherein the immune checkpoint targeting feature inhibits an immune checkpoint in a tumor or cancer cell; and
applying light activation to a region of the patient in proximity to the tumor, whereby the photoactivatable nanoparticles are self-delivered to the tumor, and whereby the photoactivatable nanoparticles inhibit growth of the tumor.

10. The method of claim 9, wherein the nanoparticles are liposomes, solid lipid nanoparticles, organic or inorganic nanoparticles, self-assembled nanoparticles, crystalline nanoparticles, amorphous nanoparticles, lipid micelles, polymer micelles, hybrid micelles, or combinations thereof.

11. The method of claim 9, wherein the photosensitizer is BPD-PC.

12. The method of claim 9, wherein the immune checkpoint targeting feature is an α-PD-L1 antibody.

13. The method of claim 9, wherein the light activation is applied at 690 nm.

14. The method of claim 9, wherein the nanoparticles are liposomes, wherein the photosensitizer is BPD-PC, wherein the immune checkpoint targeting feature is an α-PD-L1 antibody, and wherein seventeen α-PD-L1 antibodies are attached to each nanoparticle.

15. The method of claim 9, further comprising the step of administering one or more additional treatments for inhibiting tumor growth to the patient.

16. The method of claim 15, wherein the one or more additional treatments are chemotherapy, small molecular weight inhibitors, targeted drugs, nucleic acids, mRNA, DNA, radiosensitizers, or combinations thereof.
